(19) United States
(12) Patent Application Publication
(10) Pub. No.: US 2024/0299513 A1
(43) Pub. Date: Sep. 12, 2024

(54) NOVEL mRNA VACCINE FOR AUTOIMMUNITY

(71) Applicant: The Trustees of Columbia University in the City of New York, New York, NY (US)

(72) Inventors: Remi J. Creusot, New York, NY (US); Rebuma Firdessa Fite, Fort Lee, NJ (US); Jorge Postigo Fernandez, New York, NY (US)

(21) Appl. No.: 18/572,241

(22) PCT Filed: Jun. 22, 2022
(86) PCT No.: PCT/US22/73087

§ 371 Date: Dec. 20, 2023

Related U.S. Application Data
(60) Provisional application No. 63/202,741, filed on Jun. 22, 2021.

(51) Int. Cl. A61K 39/00; A61P 3/10

(52) U.S. Cl. CPC A61K 39/0008; A61P 3/10; A61K 2039/53; A61K 2039/55555

(57) ABSTRACT

This disclosure describes a nucleic acid construct that contains sequences for an Endotope construct, a STAT1c, and miR142 target sites. In one example, disclosed is composition comprising an Endotope construct and a STAT1 construct including a nucleic acid sequence encoding a constitutively active STAT1 (e.g. STAT1c), wherein the Endotope and the STAT1 constructs each include miR142 target sites. In alternative examples, disclosed is a single construct that includes the Endotope construct and STAT1 construct along with miR142 target sites. The nucleic acid constructs can be packaged into polycationic molecules or liposome to create nanoparticles for efficient cell transfection.

### CROSS-REFERENCE TO RELATED APPLICATION

This application claims benefit of United States Provisional Patent Application No. 63/202,741, filed Jun. 22, 2021, titled “NOVEL mRNA VACCINE FOR AUTOIMMUNITY”, which is incorporated herein in its entirety.

### GOVERNMENT STATEMENT CLAUSE

This invention was made with government support under AI110812 awarded by the National Institutes of Health. The government has certain rights in the invention.

### BACKGROUND

Autoimmunity occurs as a consequence of adaptive immune responses against self-antigens (self Ags) expressed in specific tissues. For example, Type 1 diabetes (TID) results from the autoimmune destruction of insulin-producing B-cells by B-cell Ag-reactive diabetogenic T cells. TID affects several millions of Americans and its incidence inexorably increases each year¹⁻³. There is no cure and life-long insulin replacement with exogenous insulin does not preclude severe complications. Ag-specific immunotherapies (ASITs), unlike non-ASIT therapies^{4.5}, aim to target and disarm the disease-causing lymphocyte populations, without affecting other immune cells and jeopardizing our overall immune protection. Several ASIT and non-ASIT strategies have been investigated clinically^{5.6} but there is still no FDA-approved therapy for TID. ASITs involve delivery of autoantigens, under various forms and routes, with the aim to desensitize (tolerize) T cells reactive to these Ags. However, the nature of the Ag-presenting cells (APCs) involved is usually not known or not well-controlled.

Hematopoietic cells, particularly dendritic cells (DCs), play a dual role in regulating immunity. Depending on conditions, they can elicit T cell immunity (‘fight signal’ or immunogenic) or T cell tolerance (‘stand down signal’ or tolerogenic). When autoantigens are presented by immunogenic DCs, they may negate the effect of tolerogenic APCs or even exacerbate disease. Moreover, DCs have numerous reported alterations in TID that cause them to be less tolerogenic⁷. In contrast, non-hematopoietic (stromal) cells encompass a wide variety of cell types that do not normally serve as APCs, but have yet the ability to do so under certain circumstances⁸. As these cells lack the costimulatory molecules needed to fully activate T cells, expressing various types of inhibitory molecules instead, they consistently induce tolerance in one form or another^{8,9}. Lymph node stromal cells (LNSCs), which continuously interact with T cells, have been shown to induce tolerance to Ags that they endogenously express⁹⁻¹⁵. Although they express lower MHC levels than DCs, they can still mediate deletion of CD8+ T cells via MHC-¹⁹⁻¹⁵, and also contribute to protection from autoimmunity via MHC-II^{16,17}. It was reported that mouse and human LNSCs appear more tolerogenic in pancreatic lymph nodes during TID¹⁸, but they lack the DC's ability to capture Ags.

### BRIEF DESCRIPTION OF THE DRAWINGS

The present embodiments are illustrated by way of example, and not by way of limitation, in the figures of the accompanying drawings.

The following figures are illustrative only, and are not intended to be limiting

FIG. 1A is an example of Endotope construct used for NOD mouse treatment (BDC2.5 Ag in blue, NY8.3 Ag in green).

FIG. 1B is a bar graph showing cell types transfected by mRNA-nanoparticles (NPs) in lymph node after intraperitoneal (i.p.) delivery.

FIG. 1C through FIG. 1D are graphs showing the incidence of disease in NOD mice treated with Endotope mRNA-NPs (C) or Endotope mRNA-DCs (D). Sequences related to the noted components (epitopes), are provided in U.S. Pat. No. 10,238,741 incorporated herein in its entirety.

FIG. 2 are graphs showing the effect of IFNγ and STAT1c on mouse and human fibroblastic reticular cells (FRCs), a type of LNSC. Cells were analyzed 3-4 days after IFNα or IFNγ treatment (10 ng/mL) or STAT1c transduction. Human STAT1c, was used for both cells, which may explain why it had a more pronounced effect on human FRCs.

FIG. 3A-FIG. 3B are graphs showing the phenotype of different types of LNSCs (FRCs and lymphatic endothelial cells, LECs) with and without exposure to IFNγ (FIG. 3A) and their ability to engage Ag-specific T cells based on epitopes they express under these conditions (FIG. 3B). FIG. 3(A) Expression of markers Pdpn and CD31 characterizing FRCs and LECs among CD45-stromal cells. Expression of MHC class I and II, PD-L1 and CD86 at baseline or after 3 days of treatment with IFNα or IFNγ. FIG. 3(B) Untransduced cells or cells transduced with Endotope construct including epitopes for BDC2.5 and NY8.3 T cells, with or without IFNγ pretreatment were co-cultured with BDC2.5 CD4+ T cells and/or NY8.3 CD8+ T cells. T cell responses were analyzed 3 days later (here shown the co-induction of CD25 and Lag-3 as evidence of productive engagement).

FIG. 4A-FIG. 4E are schematic representations of the tolerogenic mRNA vaccine strategy. FIG. 4A) Schematic representation of modified mRNA encoding Endotope Ags and STAT1c with incorporated miR142T in nanoparticle formulation. FIG. 4B) When mRNA/NPs transfect hematopoietic APCs (e.g. DCs), the mRNA is targeted by miR142 and degraded; Ags are not expressed and these APCs do not engage autoreactive T cells. FIG. 4C) When mRNA-NPs transfect stromal cells, the mRNA is expressed and produces MHC class I and II-targeted epitopes and STAT1c. FIG. 4D) STAT1c stimulates IFNγ-responsive genes, upregulating MHC class I and II, PD-L1, IDO and iNOS but not costimulatory molecules, turning these stromal cells into effective tolerogenic APCs. FIG. 4E) Autoreactive T cells are engaged by reprogramed stromal cells and themselves reprogramed to shut down or undergo apoptosis.

FIG. 5A-FIG. 5F Validation of cell selective expression with miR-142T. Lymph node stromal APCs include human fibroblastic reticular cells (FRCs) in vitro (FIG. 5A), mouse FRCs in vitro (FIG. 5B) and mouse blood endothelial cells (BEC) in vivo (FIG. 5C). Hematopoietic APCs included human monocytic THP-1 cells (FIG. 5D), mouse bone marrow-derived dendritic cells (BM-DCs) (FIG. 5E) and mouse CD11c+CD11b+cDC2 cells in vivo (FIG. 5F). FRCs were transfected with 0.1 ug mRNA/well and analyzed after 48 h. THP-1 cells and BM-DCs were transfected with 0.2 ug mRNA/well and analyzed after 48 h (THP-1) or 24 h (BM-DCs). For all experiments, mRNA was complexed in NPs using in vivo JetRNA (Polyplus). Mice were injected intraperitoneally with 20-22.4 ug mRNA/mouse, and pancreatic lymph nodes and spleen were processed and digested for analysis after 48 h.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated by reference.

Generally, nomenclatures used in connection with, and techniques of, cell and tissue culture, molecular biology, immunology, microbiology, genetics, protein, and nucleic acid chemistry and hybridization described herein are those well-known and commonly used in the art. The methods and techniques of the present invention are generally performed according to conventional methods well known in the art and as described in various general and more specific references that are cited and discussed through the present specification unless otherwise indicated.

The terms “administering” or “administration” of an agent, drug, or peptide to a subject refers to any route of introducing or delivering to a subject a compound to perform its intended function. The administering or administration can be carried out by any suitable route, including orally, intranasally, parenterally (intravenously, intramuscularly, intraperitoneally, intradermally or subcutaneously), rectally, or topically. Administering or administration includes self-administration and the administration by another.

An “antigen,” or “Ag” as the term used herein, is a structural substance (molecule or chemical group), often a protein, or peptides derived from this protein, that is recognized by the immune system of an organism and serves as a target for an immune response.

A “self Ag” or “autoantigen” is an Ag which under normal circumstances is not immunogenic and does not produce an immune response, but which may become a target of an immunogenic immune response, resulting in an autoimmune disease. For purposes of the disclosed constructs, it is noted that self Ag may be derived from autologous or allogenic source.

“Autoimmune diseases” are caused by abnormal immune responses against self Ags, wherein the immune system attacks normal tissues or organs. Type 1 diabetes is an autoimmune disease where cells that recognize insulin or other beta-cell Ags have become activated and destroy pancreatic beta cells, leading to diabetes.

The term “constitutively active” as used herein with respect to a protein means that the protein is always functionally active.

The term “construct,” as used herein, refers to a nucleic acid which encodes a protein or peptide of interest, and optionally contains one or more promoters for expression of that protein or peptide in a cell.

The term “Endotope” refers to a nucleic acid construct engineered for modulating the immune system that optimizes presentation of CD4 and CD8 epitopes by APCs in which the construct has been introduced by way of transfection or transduction. Epitopes from either self Ags or non-self Ags (e.g. tumor or pathogen-derived Ags) can be used to optimize either the induction of tolerance or immunity to those epitopes, respectively. An Endotope construct may include respective nucleic acid sequences that encode one or more CD4 epitopes targeted for MHCII processing within the endosomes of a cell and one or more CD8 epitopes targeted for

MHCI processing within the cytosol of the cell, to produce the maximum Ag/epitope presentation in the immune system, and may further include an MHCII activator sequence. Alternatively, the constructs encode CD4 and CD8 epitopes operably linked to a secretion signal. The constructs of this disclosure are intended to facilitate a greater involvement of stromal cells (SCs) to effectively engage and reprogram self-reactive T cells to achieve or reinstate tolerance. Particularly exemplified herein is endogenous delivery of epitope-expressing constructs in the form of DNA or RNA vaccines to non-professional APCs, such as SCs. In certain embodiments, Endotope constructs can contain two groups of linked epitopes, where the groups are separated from each other by a proteolytic cleavage site, and where one group of epitopes destined for processing in the MHCII pathway for presentation to CD4+ T cells (CD4 epitopes) is operably linked to an endosomal targeting sequence and the other group of epitopes destined for processing in the MHCI pathway for presentation to CD8+ T cells (CD8 epitopes) does not. After cleavage at the proteolytic cleavage site, the two groups of epitopes in the Endotope construct are separated so that each group undergoes separate processing within the cell, one onto MHCII and the other one onto MHCI. Each epitope in each of the two groups may be separated by a proteolytic cleavage site so that once in the appropriate cellular compartment for processing, each epitope is cleaved from the other epitopes in the group.

An “epitope,” (also referred to as an “antigenic determinant”) as the term is used herein, is the part of an Ag which is specifically recognized by the immune system. Epitopes are either in the form of peptides presented by MHC molecules and recognized by T cell through their T cell receptor, or correspond to exposed regions of a complete Ag that are recognized by B cells through their B cell receptor, and later by antibodies that these B cells produce. The term epitope is interpreted to include mimotopes unless indicated otherwise or if the context of the reference implies that only natural epitopes are being described.

The term “MHCII activator sequence” refers to a sequence that induces production of MHCII molecules when expressed in a cell. One non-limiting example of an MHCII activator sequence is the Class II TransActivator (CIITA) sequence (see Kim et al., J Immunol 2008; 180:7019-7027).

As used herein, a “mimotope” is a molecule that mimics the three-dimensional structure of an epitope, and therefore has the same or a highly similar binding specificity, but may or may not have a different affinity or avidity. A “mimotope” causes an antibody response similar to that elicited by the epitope which it mimics. An antibody elicited against a particular epitope (Ag) recognizes a mimotope of that particular epitope, and a mimotope of a particular epitope can elicit an antibody response which binds that particular epitope. Therefore, one or more mimotopes can be used as a vaccine. A mimotope may be, as are most epitopes, a portion of a macromolecule, such as a protein, nucleic acid or polysaccharide. Preferably, it is a protein or a portion of a protein, and may be a peptide typically about 9 to about 20 amino acids in length. Some mimotopes may appear as mutants of naturally occurring epitopes, whereby the “mutation” in fact represents an amino acid change resulting from a post-translational modification (for example, deamidation, resulting in changes from Asn to Asp and Gln to Glu) that may naturally occur under certain pathogenic conditions. Mimotopes are either obtained by screening phage-display or peptide libraries, or by directed mutagenesis aimed at altering the binding properties of the peptide, according to methods known in the art.

“Stromal cells” (SCs) as used herein refers to cells that are part of the stroma. SCs are connective tissue cells of an organ and support the function of the parenchymal cells of that organ. SCs can include fibroblasts and pericytes, their precursors mesenchymal stromal cells, as well as certain types of endothelial and epithelial cells. In certain embodiments, stromal cells are lymph node stromal cells (LNSCs), which have the particularity of being constantly in direct contact with immune cells.

The term “professional antigen presenting cells” or “professional APCs” as used herein refers to dendritic cells, macrophages and B cells.

“T cells” are a type of lymphocytes. T helper cells (CD4+ T cells) become activated when they are presented with peptide Ags by MHCII molecules on the surface of APCs. Once activated, T helper cells divide and secrete cytokines that stimulate an active immune response. Some T helper cells conversely differentiate to become regulatory, with the ability to suppress adaptive immune responses. Cytotoxic T cells (CD8+ T cells) are activated by binding to Ag associated with MHCI molecules on the surface of APCs, and destroy virus-infected cells and tumor cells. These cytotoxic T cells also are implicated in transplant rejection and autoimmune damage. A self-reactive (or autoreactive) T cell is a CD4+ or CD8+ T cell that is or has been activated by a self Ag (or autoantigen).

“Secretion signal” is a peptide that when operably linked to one or more epitopes directs secretion of the one or more epitopes out of the cells in which they are expressed.

As used herein, “therapeutically effective amount” or “an effective amount” have the standard meanings known in the art and are used interchangeably herein to mean an amount sufficient to treat a subject afflicted with a disease (e.g., diabetes) or to alleviate a symptom or a complication associated with the disease.

The terms “miR142” or “MicroRNA 142” refers to an RNA Gene and is affiliated with the miRNA class. Diseases associated with miR142 include brain cancer and multiple sclerosis. Hematopoietic cells express miR142, but not stromal cells (25). The term “miR142 target site (miR142T)” refers to any nucleic acid sequences that is complementary to miR142 or sequences that miR142 can bind to. In the context of the present invention, the binding of miR142 to a miR142T present on a construct results in the degradation of the construct-encoded mRNA and/or suppression expression of any protein/peptide sequences encoded by nucleic acid sequences on the construct. Constructs that include miR142 target sites (miR142T) downstream of introduced genes can be expressed in hepatocytes (27,26) and stromal cells (15) but not in hematopoietic cells because the transcribed mRNA is degraded by miR142 before it can be expressed.

The terms “nucleic acid,” “polynucleotide,” and “oligonucleotide” are used interchangeably and refer to a deoxyribonucleotide or ribonucleotide polymer, in linear or circular conformation, and in either single-or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T.

The terms “polypeptide,” “peptide” and “protein” are used interchangeably to refer to a polymer of amino acid residues. The term also applies to amino acid polymers in which one or more amino acids are chemical analogues or modified derivatives of a corresponding naturally-occurring amino acids.

The terms “pharmaceutically acceptable carrier, excipient, vehicle, or diluent” refer to a medium which does not interfere with the effectiveness or activity of an active ingredient and which is not toxic to the hosts to which it is administered. A carrier, excipient, vehicle, or diluent includes but is not limited to binders, adhesives, lubricants, disintegrates, bulking agents, buffers, and miscellaneous materials such as absorbents that may be needed in order to prepare a particular composition.

“Polycationic molecule,” as used herein, refers to a positively charged molecule that when complexed to a nucleic acid construct induces its condensation into a more compact macromolecule and increases capture by cells. Transfection can be achieved in all types of cells, though at variable efficiency. Stromal cells and certain parenchymal cells that replicate more frequently tend to be transfected a lot more efficiently than professional APCs, which tend to degrade DNA or mRNA complexes before the DNA or mRNA has a chance to escape from endosomes to cytosol. Polycationic molecules include small non-immunogenic peptides comprised of positively charged amino acids, such as polyarginine, poly-L-lysine and the HIV-based Tat peptide (GRKKRRQRRRPQ SEQ ID NO:18). In a preferred embodiment, the CPP are polycationic lipids. Polycationic lipids have the ability to form aggregate complexes with negatively charged genetic material such as DNA or RNA. These aggregated liposomal structures have a positive surface charge when in aqueous solutions. Polycationic lipids can safely deliver nucleic acids in vivo to target a wide range of tissues, through various routes of administrations. These peptides are typically referred to as “cell-penetrating peptides” (CPP). Other polycationic molecules include positively charged polymers such as polyethylenimine (PEI) and polyamidoamine (PAMAM). These polycationic molecules are described in more details elsewhere (Non-viral vectors for gene-based therapy. Yin H, Kanasty R L, Eltoukhy A A, Vegas A J, Dorkin J R, Anderson D G. Nat Rev Genet. 2014 August; 15(8):541-55). Upon endocytosis of the complexed nucleic acid construct, where there is a low pH inside the endosome, protons neutralize the negative charges of the nucleic acid sequence, and the polycationic molecules detach and can disrupt the membrane of the endosome.

A “sequence,” as used herein, refers to the primary structure of a biological macromolecule or oligomolecule, or the ordering of monomers (nucleotides or peptides, for example) covalently linked within a biopolymer.

The term “sequence identity” or “identity,” as used herein in the context of two polynucleotides or polypeptides, refers to the residues in the sequences of the two molecules that are the same when aligned for maximum correspondence over a specified comparison window. As used herein, the term “percentage of sequence identity” or “% sequence identity” refers to the value determined by comparing two optimally aligned sequences (e.g., nucleic acid sequences or polypeptide sequences) of a molecule over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleotide or amino acid residue occurs in both sequences to yield the number of matched positions, dividing the number of matched positions by the total number of positions in the comparison window, and multiplying the result by 100 to yield the percentage of sequence identity. A sequence that is identical at every position in comparison to a reference sequence is said to be 100% identical to the reference sequence, and vice-versa.

The term “STAT1” or “Signal transducer and activator of transcription 1” refers to a transcription factor which in humans is encoded by the STAT1 gene. Non-limiting examples of STAT1 genes include SEQ ID NOs 1 or 2). STAT1 includes any of SEQ ID NOs 3-8, or an amino acid sequence having at least 95 percent identity therewith. It is a member of the STAT protein family. All STAT molecules are phosphorylated by receptor associated kinases, that causes activation, dimerization by forming homo- or heterodimers and finally translocate to nucleus to work as transcription factors. Specifically, STAT1 can be activated by several ligands such as Interferon alpha (IFNα), Interferon gamma (IFNγ), Epidermal Growth Factor (EGF), Platelet Derived Growth Factor (PDGF), Interleukin 6 (IL-6), or IL-27. STAT1 is involved in upregulating genes due to a signal by either type I, type II, or type III interferons. In response to IFN-γ stimulation, STAT1 forms homodimers or heterodimers with STAT3 that bind to the GAS (Interferon-Gamma-Activated Sequence) promoter element; in response to either IFN-α or IFN-β stimulation, STAT1 forms a heterodimer with STAT2 that can bind the ISRE (Interferon-Stimulated Response Element) promoter element. In either case, binding of the promoter element leads to an increased expression of ISG (Interferon-Stimulated Genes).

The term “subject” as used herein refers to an individual. For example, the subject is a mammal, such as a primate, and, more specifically, a human. The term does not denote a particular age or sex. Thus, adult and newborn subjects, whether male or female, are intended to be covered. As used herein, patient or subject may be used interchangeably and can refer to a subject afflicted with a disease or disorder.

The term “target site” as used herein refers to the nucleic acid sequence or region that is recognized (e.g., specifically binds to) and/or acted upon (excised or cut) by a microRNA, siRNA, or shRNA.

“Immune tolerance” as used herein is the mechanism of non-self discrimination which allows the immune system to recognize foreign Ags, but not self Ags. Under normal conditions, tissue-specific self Ags are presented by tolerance-inducing (tolerogenic) cells, which program T cells not to respond to these Ags. Autoimmune disease results when these self Ags are not tolerized.

### DETAILED DESCRIPTION

It was found that stromal cells can be reprogrammed into more efficient APCs by overexpression of STAT1c (a mutated form of STAT1 that results in constitutive activity of STAT1 by dimerization in absence of phosphorylation). The reprogrammed stromal cells then can be made to express Endotope constructs and Ags to optimize the engagement of both CD4 and CD8 T cells. However, it has also been shown that broad delivery of Ags to both stromal cells and professional APCs may result in defective tolerogenic potential. It was found that miR142 is not expressed in non-hematopoietic cells such as hepatocytes and stromal cells but is expressed in hematopoietic cells, which include professional APCs. Administration of an Endotope construct that includes an miR142 target site sequence, and optionally in combination with a sequence encoding STAT1c (either on the same construct or separate construct) allows for the selective expression of the Endotope-encoded peptides in stromal cells and not in professional APCS. This selective expression enhances tolerogenic immune responses to an Ag or epitopes of interest.

This disclosure describes a nucleic acid construct that contains sequences for an Endotope construct, a STAT1c, and miR142 target sites. In certain embodiments, disclosed is composition comprising an Endotope construct and a STAT1 construct including a nucleic acid sequence encoding a constitutively active STAT1 (e.g. STAT1c), wherein the Endotope and the STAT1 constructs each include miR142 target sites. Alternatively, disclosed is a single construct that includes the Endotope construct and STAT1 construct along with miR142 target sites. The nucleic acid constructs can be packaged into polycationic molecules to create nanoparticles for efficient cell transfection. The Endotope construct is customizable and can deliver patient-specific or highly shared epitopes that are disease-relevant. Certain embodiments provided herein relate to a method for treating autoimmune disorders by administering the novel nucleic acid constructs in the form of a DNA or RNA vaccine.

### Overview

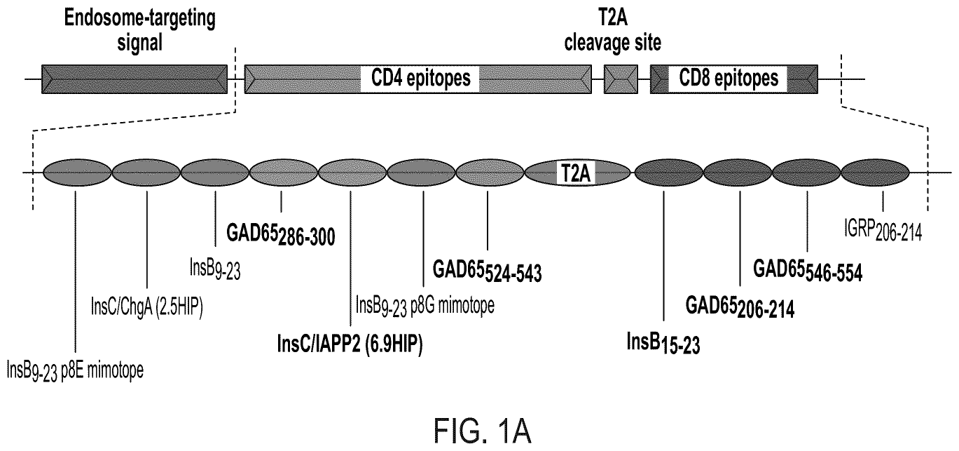

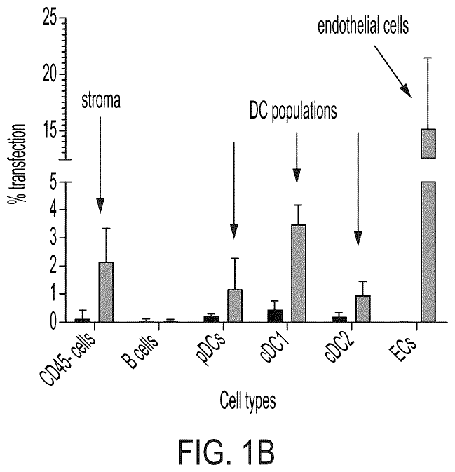

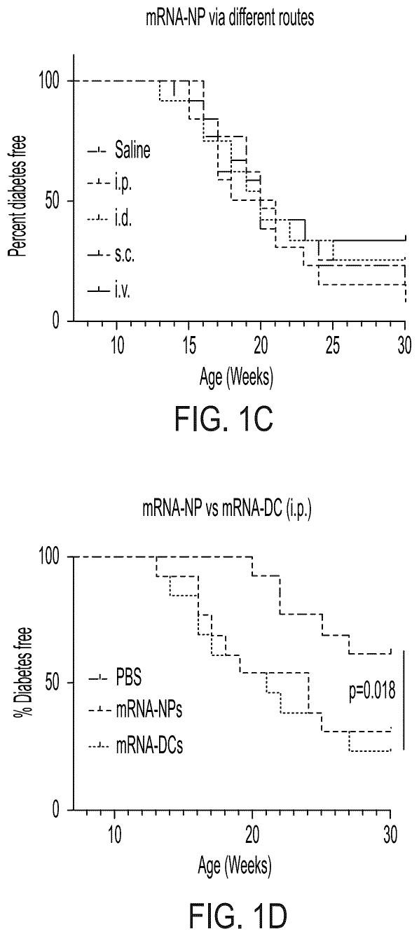

A platform (Endotope) was developed for nucleic acid-based delivery of select epitopes to engage specific T cell populations that are major drivers of a disease (FIG. 1A as example). The patented design of Endotope enables efficient presentation of endogenously expressed epitopes on MHC-I and MHC-II as appropriate, resulting in optimal engagement of both CD4 and CD8 T cells¹⁹. This platform is used for DNA vaccines²⁰, nanoparticle-formulated mRNA vaccines (mRNA-NP)^{21.22} and also introduced them by mRNA electroporation or transduction into DCs or stromal cells ex vivo^{19,23}. The mRNA-NP delivery system can target both LNSC and DC subsets²¹ (FIG. 1B), but when Ags were delivered by mRNA-NP to NOD mice by various routes, they failed to ameliorate disease (FIG. 1C), while injection of tolerogenic DCs electroporated with the same mRNA significantly reduced disease incidence (FIG 1D).

This suggests that broad delivery of Ags to various APCs, which may include immunogenic DCs in the context of an ongoing autoimmune disease (with chronic inflammation), may result in different and unwanted T cell responses. Although the mRNA is modified by nucleotide substitutions to minimize its adjuvanticity²⁴, it may still enhance the immunogenicity of some DCs or the mRNA-NPs may transfect DCs that are already in immunogenic state. Moreover, the cationic lipid formulation may have an immunogenic adjuvant effect on professional APCs (Nat Immunol. 2022 April; 23(4):532-542). Because only hematopoietic cells express the miR142 microRNA²⁵, viral vectors that feature miR142 target sites (miR142T) downstream of introduced genes can safely be expressed in non-hematopoietic cells such as hepatocytes²⁵⁻²⁷ and stromal cells¹⁵. The viral vectors will not be expressed in hematopoietic cells because the transcribed mRNA is degraded by miR142 before it can be expressed. This system is in the process of being validated in a non-viral delivery system featuring our mRNA-NP platform.

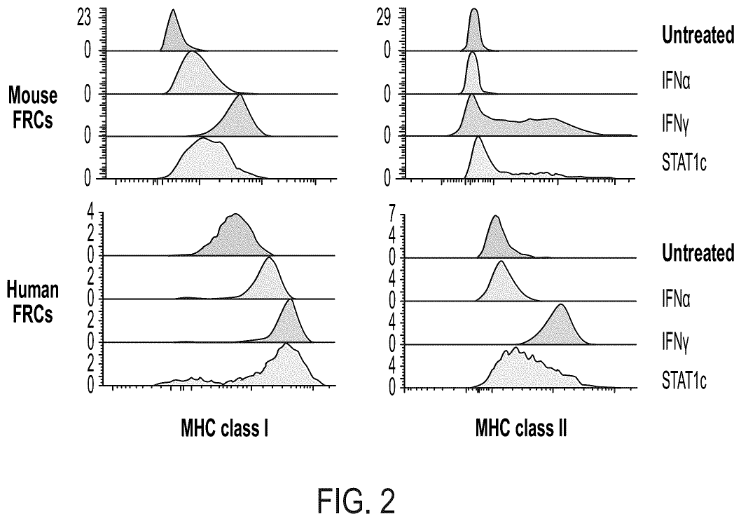

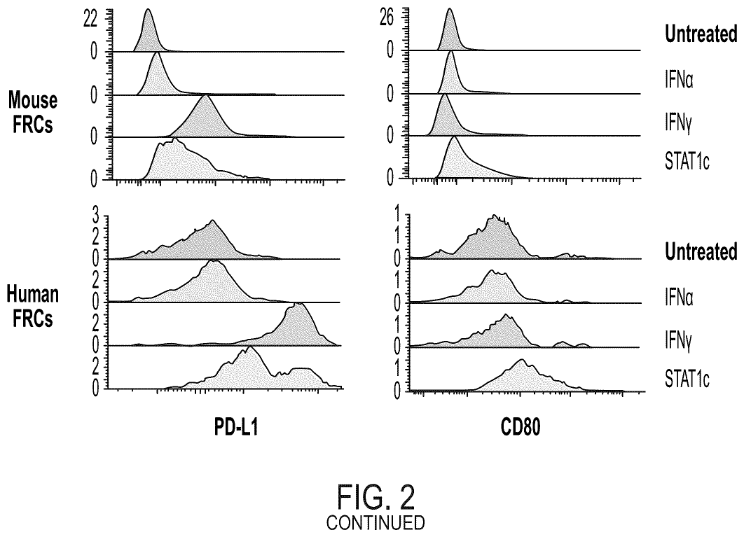

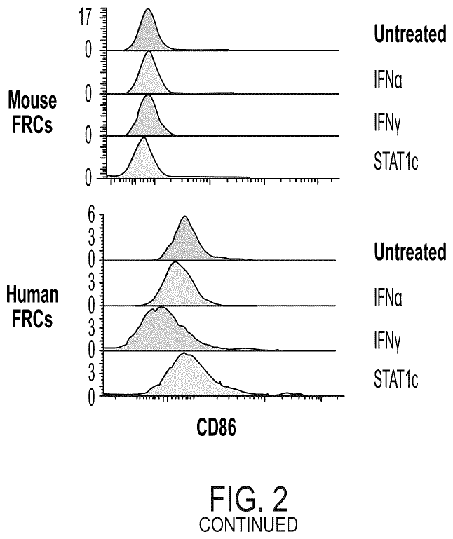

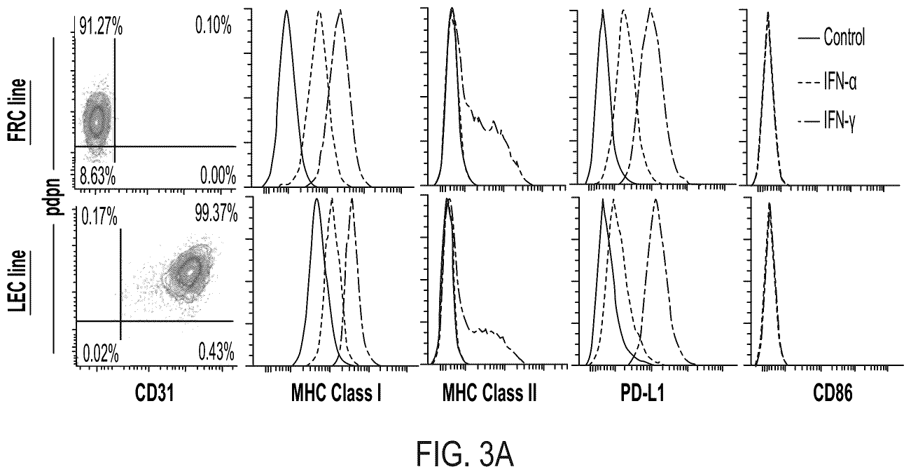

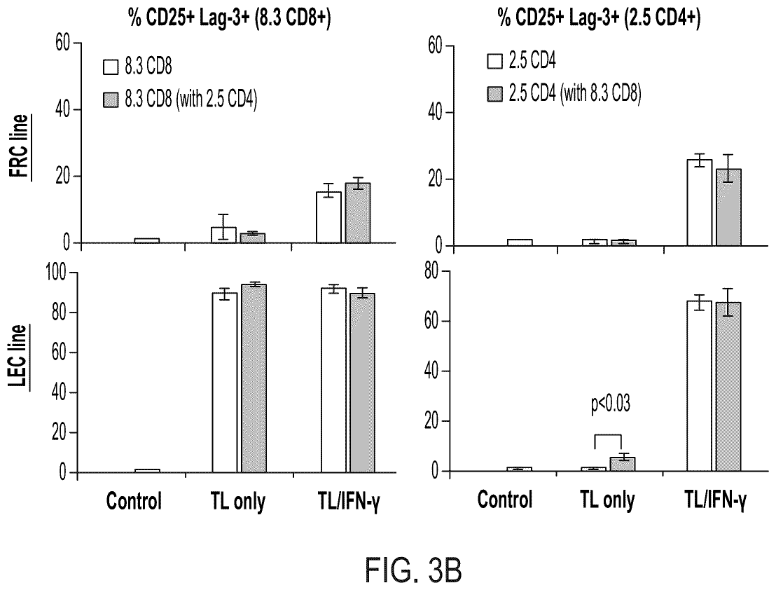

Stromal cells can be programed into more efficient and more tolerogenic APCs. IFNγ has a well-established regulatory effect on stromal cells (including mesenchymal stromal cells and LNSCs) to enhance MHC-I and MHC-II levels^{17,28-30}, as well as tolerogenic potential via PD-L1³⁰⁻³², indolcamine^{2,3}-dioxygenase (IDO)^{30,33,34} and inducible nitric oxide synthase (iNOS)^{29.35.36}, all of which have inhibitory effects on T cells. Several LNSC subsets, fibroblastic reticular cells (FRCs; FIG. 2, FIG. 3A, & FIG. 3B) and lymphatic endothelial cells (FIG. 3A. and FIG. 3B), upregulate MHC-I, MHC-II and PD-L1, but not costimulatory molecules (CD80/CD86), in response to IFNγ treatment. To reprogram these cells in vivo without the use of IFNγ, which can have unwanted effects on other immune cells, a constitutively active form of STAT1 (STAT1c) was tested that recapitulates IFNγ signaling. Overexpression of STAT1c, similar to IFNγ treatment, reprogramed these cells to be more efficient APCs (higher MHC levels to engage both CD4 and CD8 T cells) and more tolerogenic (increased PD-L1 without costimulatory molecule induction) (FIG. 2).

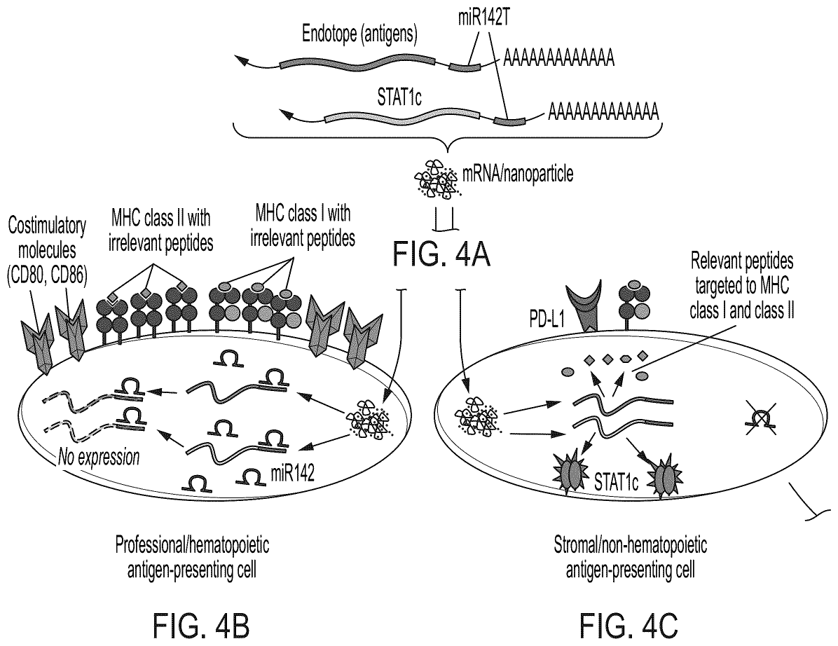

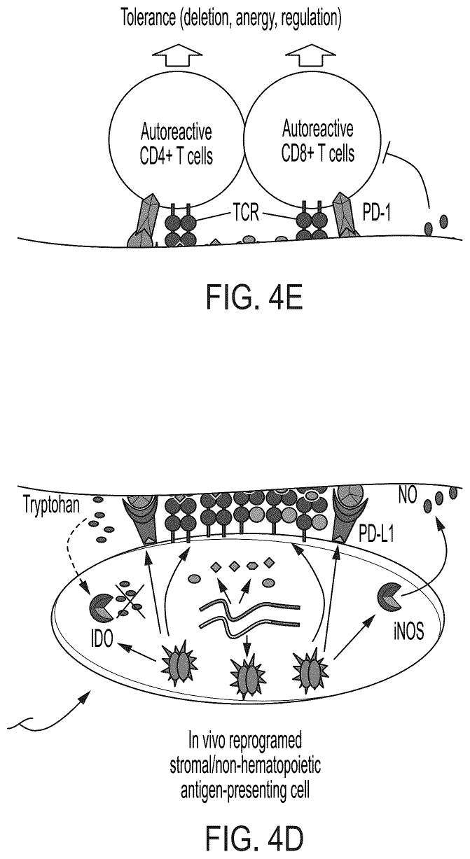

This disclosure describes a novel mRNA vaccine, whose applicability has been boosted by the recent FDA approval of mRNA vaccines against SARS-COV-2, but is unconventional in harnessing stromal cells to reprogram autoreactive T cells toward tolerance (FIG. 4). Current ASITs have several limitations: (1) most provide Ags in the form of exogenous peptides/proteins which are primarily acquired and presented by DCs, which in the case of TID have defective tolerogenic potential⁷, and (2) they do not yet include novel neoepitopes (not present in the native peptides/recombinant proteins used). This mRNA-NP approach enables endogenous expression of peptides (epitopes) that include neoepitopes or that can undergo post-translational modifications within cells. This novel vaccine features multiple innovations: (1) the Endotope design enabling presentation of mRNA-encoded epitopes to both CD4 and CD8 T cells, (2) a cationic lipid-based formulation for efficient in vivo mRNA delivery recently made commercially available²², (3) the miR142T feature to restrict expression of Ags and accessory molecules to stromal cells (preventing presentation by DCs), and (4) the use of STAT1c as accessory molecule to reprogram stromal cells as more efficient and more tolerogenic APCs to engage autoreactive T cells and shut down their response.

Advantages of mRNA include very efficient cell transfection (including quiescent stromal cells), lack of genome integration and transient expression of products, both of which are safety features. This unconventional vaccine is significant in minimizing the risk of ASITs by obviating the involvement of DCs in the presentation of delivered autoantigens, another important safety feature. Stromal cells can be made better APCs without becoming immunogenic, even with mRNA, as in vivo stimulation of LNSCs via TLR3 increases MHC-I and PD-L1 but not costimulatory molecules⁹, as seen with IFNγ. This disclosure describes an innovative and potentially safer form of ASIT for autoimmune diseases, satisfying an unmet need. Furthermore, the customizable Endotope platform constitutes an ideal tool for the delivery of patient-tailored or highly shared epitopes³⁷⁻⁴⁰ within groups of patients as a precision medicine approach to TID and several other autoimmune diseases. A large population of recent onset TID patients and individuals identified as high-risk would benefit from this new ASIT to block the autoimmune response and preserve endogenous β-cells.

### Endotope Constructs

Certain embodiments of this invention are directed to Endotope constructs carrying a fusion peptide sequence encoding an operably linked endosomal MHCII targeting sequence followed by one or more epitope sequences for CD4+ T cells (presented on MHCII), a series of one or more CD8 epitope sequences (presented on MHCI), with a cleavable linker separating the two epitope sequences and an MHCII activator sequence operably linked to the one or more epitope sequences. This type of construct, called Endotope, enables delivery, into single cells, of multiple disease-driving epitopes expressed by a single nucleic acid-based (DNA or RNA) construct or multiple mRNA molecules in the same complex that initiates immune tolerance to epitopes recognized by both autoreactive CD4+ and CD8+ T cells through optimized Ag presentation and processing and equips transfected stromal cells with the ability to present CD4 epitopes on MHCII. While the Endotope constructs carry an MHCII targeting sequence operably linked to the CD4 epitopes intended for processing in endosomes, it is not necessary to include an MHCI targeting sequence for CD8 epitopes because the construct is delivered to the cytoplasm where these epitopes will be processed via proteasomes, according to the normal cellular process.

The Endotope constructs may be codon-optimized for the species in which the construct is used. Codon optimization may include nucleotide changes that reduce or enhance the immunogenicity of the vector without altering the amino acid sequence.

In an embodiment for treatment of diabetes, for example, the Endotope construct allows targeting of both CD4+ and CD8+diabetogenic T cells for deletion or suppression across multiple beta-cell Ags, using the tolerogenic DNA vaccination strategy that has a good safety profile in TID patients (Roep et al., 2013).

The Ags encoded by the Endotope constructs can be customized not only for various diseases requiring either immune tolerance such as autoimmune diseases or immune stimulation such as infectious diseases, but can also be customized for individual patients to elicit the greatest tolerance response or the greatest immune response. Various immunoassays exist to determine whether some immune cells circulating in the blood in a given patient develop an immune response to particular peptides tested. Alternatively, the Ags selected can be based on the most common reactivity seen in a class of patients. Because it is customizable, native peptides may be mutated for better targeting of specific types of self-reactive T cells (those requiring post-translational modifications or an uncommon MHC binding register). The Endotope constructs provide a way to ensure endogenous expression of dominant disease epitopes (including modified neoepitopes) that cannot be achieved with simple administration of combined exogenous proteins. Because only the important selected disease epitopes are included in the Endotope construct, a single construct suffices to present a plurality of epitopes from multiple protein Ags to both or either CD4+ and CD8+ T cells, enabling a balanced expression of both CD4 and CD8 epitopes. In previous approaches, constructs needed to include the sequence of each entire protein, which is problematic because the capacity of constructs and vehicles to deliver nucleic acids is limited.

Below is a list of Ags that are known for certain diseases. Epitopes from these Ags can be included in the nucleic acid constructs for treatment of the respective diseases. For an overview, see Di Lorenzo et al., Clin. Exp. Immunol. 148(1):1-16, 2007 and James E A et al Diabetes. 2020 July;69(7):1311-1335. Of, interest, hybrid insulin peptides have been recently recognized as important disease-driving Ags, which are not represented in any full protein Ag (48-50).

TABLE 1A
Exemplary Autoantigens in T1D
Major Ags:
Proinsulin/insulin (gene: INS): extensive CD4+ T cell responses in NOD
mice and T1D patients, extensive CD8+ T cell in T1D patients, some
CD8+ T cell in NOD mice, autoantibodies in NOD mouse and T1D
patients.
GAD65 (glutamic acid decarboxylase, gene: GAD2): extensive CD4+ T
cell responses in NOD mice and T1D patients, some CD8+ T cell
responses in NOD mice and T1D patients, autoantibodies in T1D
patients.
IA-2 (insulinoma-associated protein 2, or protein tyrosine phosphatase,
receptor type, N, gene: PTPRN): extensive CD4+ T cell responses in
NOD mice and T1D patients (Peakman et al., J. Clin. Invest.
104(10): 1449-1457, 1999), some CD8+ T cell responses in T1D patients,
autoantibodies in T1D patients (Bonifacio et al., J. Immunol.
155(11): 5419-5426, 1995).
IGRP (islet-specific glucose-6-phosphatase catalytic subunit-related
protein; gene: G6PC2): some CD4+ T cell responses in NOD mice and
T1D patients (Jarchum et al., Clin Immunol, 127(3): 359-365, 2008),
some CD8+ T cell responses in T1D patients, extensive CD8+ T cell
responses in NOD mice.
ZnT8 (zinc transporter 8; gene: SLC30A8): autoantibodies in T1D
patients, evidence of T cell responses in NOD mice (Dang et al., J.
Immunol, 186(10): 6056-6063. 2011); (Nayak et al., Diabetes,
63(10): 3438-3448, 2014).
Hybrid insulin peptides (HIPs) that are fusions between insulin peptides
and peptides from other beta-cell Ags, including ChgA, IAPP1, IAPP2
and amylin (Delong, T, Wiles, T A, Baker, R L, Bradley, B, Barbour, G,
Reisdorph, R, et al. (2016). Pathogenic CD4 T cells in type 1 diabetes
recognize epitopes formed by peptide fusion. Science 351: 711-714).
Examples of such peptides that can be included in our constructs include
GDLQTLWSRMD (SEQ ID NO: 58), LQTLALWSRMD (SEQ ID NO:
59) and LQTLALNAARD (SEQ ID NO: 60). Similar HIPs identified
from T1D patients include GQVELGGGSSPETLI (SEQ ID NO: 61) and
GQVELGGGNAVEVLK (SEQ ID NO: 62) (Delong et al.). More
peptide fusion may be produced between insulin and ChgA by
transpeptidation that can stimulate diabetogenic T cell clones (Jin, N,
Wang, Y, Crawford, F, White, J, Marrack, P, Dai, S, et al. (2015). N-
terminal additions to the WE14 peptide of chromogranin A create strong
autoantigen agonists in type 1 diabetes. Proc Natl Acad Sci USA 112:
13318-13323).
Defective Ribosomal Product (DRiP) peptides include peptides from
transcripts that are derived from alternative and out-of-frame start
codons, often due to single nucleotide polymorphism that confers
increased risk to autoimmune disease (Nat Med. 2017 April; 23(4): 501-
507).
Minor Ags:
Chromogranin A (gene: CHGA): CD4+ T cell responses in NOD mice
(Stadinski et al., Proc. Natl. Acad. Sci. USA 107: 10978-10983, 2010;
Delong et al., Diabetes 61: 3239-3246, 2012); CD8+ T cell responses in
humanized NOD mice and T1D patients (Gottlieb et al., J. Autoimmun,
50: 38-41, 2014); (Li et al., Clin Immunol, 159(1): 63-71, 2015).
IAPP (islet amyloid polypeptide; gene: IAPP): CD4+ T cell responses in
NOD mice see Baker et al., J. Immunol, 191(8): 3990-3994, 2013; some
CD8+ T cell responses in T1D patients.
ICA69 (islet cell autoantigen; gene: ICA1): some CD4+ T cell responses
in NOD mice and T1D patients.
IA-2β (insulinoma-associated protein 2 beta or phogrin or protein
tyrosine phosphatase, receptor type, N polypeptide 2; gene: PTPRN2):
some CD4+ T cell responses in NOD mice and T1D patients.
RegII (regenerating islet II, gene: REG3A): T cell responses in NOD
mice (Gurr et al., Diabetes, 51(2): 339-346, 2002); (Gurr et al., Diabetes,
56(1): 34-40, 2007).
GPR78 (G protein-coupled receptor 78, when citrullinated; gene:
GPR78): T cell responses and autoantibodies against citrullinated GPR78
in NOD mice (Rondas et al., Diabetes, 64(2): 573-586, 2015).
HSP60
HSP70
REGII,
Vitamin D binding protein (VDBP)Autoantigens in MS
For an overview, see Riedhammer et al., Immunol. 17; 6: 322, 2015. Each of these Ags can
elicit CD4+ or CD8+ T cell responses, or both.
Major Ags:
Myelin basic protein (MBP; gene: MBP)
Proteolipid protein (PLP; gene: PLP1)
Myelin oligodendrocyte glycoprotein (MOG; gene: MOG)
Minor Ags:
Myelin-associated Ag (MAG; gene: MAG)
Myelin-associated oligodendrocyte basic protein (MOBP; gene: MOBP)
2′,3′-cyclic-nucleotide 3′-phosphodiesterase (CNPase; gene: CNP)
S100β
transaldolase.
Autoantigens in RA
See Hueber et al., Proteomic biomarkers for autoimmune disease, Proteomics, 6(14): 4100-105,
2006, and Riedhammer et al., Ag Presentation, Autoantigens, and Immune Regulation in
Multiple Sclerosis and Other Autoimmune, Diseases, Front. Immunol. 6: 322, 2015.
Major Ags:
Collagen (type II): T cell responses and antibodies
Cartilage glycoprotein 39 (Chitinase 3-like 1; gene: CHI3L1)
(Verheijden et al., Arthritis Rheum, 40(6): 1115-1125, 1997)
Aggrecan G1 (cartilage-specific proteoglycan core protein, domain G1;
gene ACAN) (Li et al., Cell Res, 10(1): 39-49, 2000)
Autoantibody Responses:
Rheumatoid factor (autoantibodies to Fc portions of IgG)
Citrullinated peptides from fibrinogen, vimentin, fillaggrin, keratin,
clusterin, biglycan, apolipoprotein E (Goronzy et al., Arthritis Res. Ther.
11(5): 249, 2009; (Sakkas et al., Autoimmun Rev. 13(11): 1114-1120,
2014) and (Wagner et al., Ann. Rheum. Dis. 74(3): 579-586, 2015).
Carbamylated Ags (Shi et al., Autoimmun Rev. 3(3): 225-30, 2014)
PAD4 and BRAF (Auger et al., Autoimmun Rev. 11(11): 801-803, 2012).
HSP65
Autoantigens in Other Autoimmune Disorders
Psoriasis and Other Skin Conditions:
Basement membrane laminin (McFadden et al., Scand J. Immunol.
10.1111-12384, 2015).
LL37 (T cell responses) (Lande et al., Nat Commun, 5: 5621, 2014).
Progranulin (autoantibodies) (Thurner et al., J. Autoimmun, 42: 29-38,
2013).
Desmoglein 3 (T cells and autoantibodies) (Nishimoto, J. Immunol,
191(6): 3065-3072, 2013).
Pso p27 (Iversen et al., Autoimmunity, 44(3): 229-234, 2011).
Ezrin, maspin, peroxiredoxin 2, HSP27 (Besgen et al., J. Immunol,
184(9): 5392-5402, 2010).
Collagen type XVII (Inokuma et al., Br. J. Dermatol, 160(2): 451-454,
2009).
Keratin 13, hnRNP-A1 and FLJ00294 (Jones et al., J. Invest. Dermatol,
123(1): 93-100, 2004).
hnRNP-C1/C2
SCG, GLCDAC05, alpha-endosulfine, NOL8, GFGR3, dematin, signal
recognition particle subunit 14 and EPF as alopecia areata autoantigens
(Lueking et al., Mol. Cell. Proteomics, 4(9): 1382-1390, 2005).
Inflammatory Bowel Disease (Crohn's Disease and Ulcerative Colitis):
Glycoproteins CUZD1 and GP2 (Roggenbuck et al., Gut, 58(12): 1620-
1628, 2009; Komorowski et al., J. Crohns Colitis, 7(10): 780-90, 2013).
HMGB1/HMGB2, ASCA autoantibodies
FAM84A (Vermeulen et al., Inflamm Bowel Dis, 17(6): 1291-1300,
2011).
Collagen type VII (Chen et al., J. Invest. Dermatol, 118(6): 1059-1064,
2002), (Hundorfean et al., J. Cell Mol Med, 14(10): 2393-2403, 2010).
Complement C3 (Lundgren et al., Eur J. Gastroenterol Hepatol,
22(4): 429-436, 2010).
Ubiquitination factor e4A (UBE4A) (Sakiyama et al., Inflamm Bowel
Dis, 14(3): 310-7, 2008).
CBir1 flagellin autoantibodies (Targan et al., Gastroenterology,
128(7): 2020-2028, 2005).
Alpha 3(IV)NC1 and BP180 autoantibodies (Plaisier et al., Am J. Kidney
Dis, 40(3): 649-654, 2002).
Galectin-3 autoantibodies (Jensen-Jarolim et al., J. Clin Immunol,
21(5): 348-356, 2001).
Catalase and alpha-enolase (Roozendaal et al., Clin Exp Immunol,
112(1): 10-6, 1998).
Lactoferrin autoantibodies (Roozendaal et al., Adv Exp Med Biol,
443: 313-319, 1998).

Any known epitope to which one would like to induce tolerance in a subject is contemplated for use with the invention. The choice of epitopes is determined based on those most often targeted in the patient population or personalized to individual patients based on diagnostic tests. The choice of epitopes is also dictated by the HLA haplotype of patients that is known to be able to present specific epitopes. The Immune Epitope Database (IEDB) represents the largest source of known epitopes, and often the MHC haplotype(s) they are known to bind to, assays related to their validation and references related to their identification. For example, a search for human epitopes in Type 1 diabetes yields ˜11,500 epitopes. Because it is customizable, native peptides may be mutated for better targeting of specific types of self-reactive T cells (those requiring post-translational modifications or uncommon MHC binding register). Preferably, the construct encodes a balance of both CD4 and CD8 epitopes so that tolerance is induced for both MHCI and MHCII Ags at the same time. Preferred Ags for making a tolerogenic nucleic acid construct include any of those specifically discussed or provided herein, or any epitopes from diabetogenic or autoimmune Ags. Thus far, three Ags have been evaluated individually in TID clinical trials (proinsulin/insulin, GAD65 and HSP60 p277) using a variety of delivery methods. Overall, these Ag-specific therapies were well-tolerated, but poorly efficacious. Other Ags are targeted in TID, including but not limited to IA-2, IGRP, ChgA and ZnT8. Given the extent of epitope spreading occurring in TID (as in other major tissue-specific autoimmune diseases), achieving tolerance against a single Ag or epitope appears to be insufficient for durable tolerance induction and may in part explain the relative failure of previous clinical trials. In addition to targeting multiple Ag specificities, efficient tolerogenic presentation to both CD4+ and CD8+ T cells, further enhanced by using mimotopes when available, increases efficiency and exploits all mechanisms of tolerance induction. Preclinical assessment of these new parameters is required in order to optimize current strategies.

A large number of epitopes from β cell Ags are now known to be targeted by diabetogenic CD4+ or CD8+ T cells in both NOD mice and TID patients, as reported in 2007 by DiLorenzo et al., with many more identified since then (see for example Delong et al. 2016, and Wang et al. 2015). The reviews by DiLorenzo et al. (Clin. Exp. Immunol. 147:doi: 10.1111/j.11365-2249.2007.03328.x (2007)) and James et al. (Diabetes. 2020 July;69(7):1311-1335. doi: 10.2337/dbi19-0022) provides sequences, examples and discussion concerning many useful T cell epitopes for autoimmune diabetes and many examples of T cell epitopes. The data presented are based on epitopes targeted in the NOD mouse model of TID, and include Ins2 B: 15-23 and IGRP206-214 for CD8 epitopes, and Ins2 B:9-23, Ins2 B:9-23 (R22E), Ins2 B:9-23 (R22E, E21G), ChgA1040-79, GAD65286-300, GAD65524-543 for CD4 epitopes/mimotopes. Some of these epitopes have been chosen for proof of principle experiments because tools and reagents exist to assess the T cell responses to these particular epitopes, such as T cell receptor transgenic mice and MHC tetramer reagents. For humans, key epitopes include those known in the field from insulin, GAD65, and IA-2. A smaller number of epitopes have been identified for other Ags, including IGRP, ChgA, ZnT8, IAPP and ICA69 as well as a number of newly discovered hybrid insulin peptides (Delong et al. 2016; James et al., 2020) and DRIP peptide (Nat Med. 2017 April; 23(4):501-507). Epitopes from HSP60/70 proteins are also targeted although those are not beta cell-specific. A widely recognized important epitope for TID is the CD4 epitope insulin B:9-23, which is targeted in both NOD mice and TID patients. In NOD mice, this epitope is involved in initiation of disease (Nakayama et al., Nature. 35(7039):220-3, 2005). There are various mimotopes designed for this epitope, which are efficacious both in mice (Daniel et al. Exp Med. 2011 Jul. 4;208(7): 1501-10), humanized mice (Serr et al., Nat Commun. 2016 Mar. 15; 7:10991) and in humans (Nakayama et al., Proc. Natl. Acad. Sci. USA 112(14):4429-34, 2015). Epitopes and mimotopes continue to be identified in a regular basis for TID and other autoimmune diseases, and will therefore complete the arsenal of epitopes already existing. As more epitopes become known, and sensitive assays that help determine which epitopes need to be targeted in particular disease or even in a particular individual, constructs and methods can be designed accordingly.

Other autoantigens, in diseases such as MS, RA, IBD, and psoriasis, some of which are listed herein, and others also known in the art, are contemplated for use with the invention. Because of the phenomenon of epitope spreading, the numbers of known autoimmune epitopes are growing. Any self Ags, and Ags from an organ or tissue to be transplanted, are also contemplated. Thus, any epitopes that become known in the future also are contemplated for use with the invention. Mimotopes can be substituted for any epitope when available. Any of the mimotopes to relevant Ags/epitopes which are known in the art can be used.

For immune tolerance, the DNA/RNA vehicles that carry the Endotope construct can be modified to remove certain motifs that are immunogenic, for example CpG motifs, which can be replaced with GpG motifs, or U-rich regions of mRNA, which can be replaced by pseudouridine.

It will be recognized that one or more features of any embodiment disclosed herein may be combined and/or rearranged within the scope of the invention to produce further embodiments that are also within the scope of the invention.

### Epitope and Construct Sequences

FIG. 1 sets forth an exemplary Endotope construct that contains a number of epitope sequences related to TID. Table 1B provides sequences of noted component sequences.

TABLE1B

| peptide | antigen | sequence | MHC | T-cell | TCR | Mouse | SEQ ID NO: |
|---|---|---|---|---|---|---|---|
| B:9-23 | Ins2 | SHLVEALYLVCGERG | I-A^{g7} | CD4 | Vβ2 | BDC12-4.1 | 11 |
| B:15-23 | Ins2 | LYLVCGERG | | CD8 | Vβ6Vα8 | G9C8 | 12 |
| B:9- | mimotope | SHLVEALYLVCGEEG | I-A^{g7} | CD4 | Vβ2 | BDC12-4.1 | 13 |
| 23(R22E) | | | | | | | |
| WE14 | ChgA | WSRMDQLAKELTAE | I-A^{g7} | CD4 | Vβ4Vα1 | BDC2.5 | 14 |
| 1040-79 | mimotope | AVPPLWVRME | I-A^{g7} | CD4 | Vβ4Vα1 | BDC2.5 | 15 |
| 206-214 | IGRP | VYLKTNVFL | K^{d} | CD8 | Vβ8.1 Vα1 | NY8.3 | 16 |
| 286-300 | GAD65 | KKGAAALGIGTSVI | I-A^{g7} | CD4 | Vβ1Vα4.5 | G286 | 17 |

Provided below as SEQ ID NO: 27 is an exemplary Endotope sequence As shown in FIG. 4, an Endotope sequence may be linked to an miRNA targeting sequence, such as miR14?T.

(SEQ ID NO: 27)
ACTAGTGCCA CCATGATGGA TCAAGCTAGA TCAGCATTCT
CTAACTTGTT TGGTGGAGAA CCATTGTCAT ATACCCGGTT
CAGCCTGGCT CGGCAAGTAG ATGGCGATAA CAGTCATGTG
GAGATGAAAC TTGCTGTAGA TGAAGAAGAA AATGCTGACA
ATAACACAAA GGCCAATGTC ACAAAACCAA AAAGGTGTAG
TGGAAGTATC TGCTATGGGA CTATTGCTGT GATCGTCTTT
TTCTTGATTG GATTTATGAT TGGCTACTTG GGCTATTGTA
AAGGGGTAGA ACCAAAAACT GAGTGTGAGA GACTGGCAGG
AACCGAGTCT CCAGTGAGGG AGGAGCCAGG AGAGGACTTC
CCTGCACCGC GGTGTGGTTC CCACCTGGTG GAGGCTCTCT
ACCTGGTGTG TGGGGAGGAG GGCTTCTTCA AGCGAGCAGT
TCGACCTCTA TGGGTACGTA TGGAAAAGGG GTCTCTCAAG
AAGGGAGCTG CAGCCTTAGG GATTGGAACA GACAGTGTGA
TTCTGATTGA GGGCAGAGGA AGTCTGCTAA CATGCGGTGA
CGTCGAGGAG AATCCTGGAC CTGAGGCTCTCTACCTGGTG
TGTGGGGAGC GTGGCTTCTT CTTGAGTGTG TACCTGAAGA
CCAACGTCTT CCTCTTCCTG CCCGGGTAGG TCGAC

TABLE 2

| Sequence Name | Sequence Location |
|---|---|
| Kozak sequence | 7-12 |
| Start codon | 13-15 |
| endosome-targeting sequence | 16-356 |
| (TFR1-118) | |
| Ins2B: 9-23 R22E mimotope | 379-423 |
| ChgA 1040-79 mimotope | 436-465 |
| GAD65286-300 native peptide | 478-522 |
| cleavage site (T2A) | 529-582 |
| Ins2B: 15-23 native peptide | 589-615 |
| IGRP206-214 native peptide | 628-654 |
| stop codon | 667-669 |

### Restriction Sites are Underlined

The codon-optimized DNA sequence of this Endotope construct is below (SEQ ID NO: 28).

(SEQ ID NO: 28)
ACTAGTGCCA CCATGATGGA CCAAGCTAGA TCCGCCTTCA
GCAATCTGTT CGGAGGAGAG CCCCTCTCCT ATACAAGATT
CTCCCTGGCC AGGCAAGTGG ACGGCGACAA CTCCCACGTC
GAGATGAAAC TCGCCGTGGA TGAAGAGGAG AACGCCGACA
ATAACACCAA GGCCAACGTG ACCAAGCCTA AGAGGTGGAG
CGGAAGCATC TGCTACGGCA CAATCGCCGTG ATCGTCTTC
TTCCTGATCG GATTCATGAT CGGATACCTG GGCTACTGCA
AGGGCGTGGA GCCTAAAACC GAGTGCGAGA GACTCGCTGG
AACAGAGTCC CCTGTCAGGG AGGAACCTGG AGAGGATTTC
CCTGCCCCGC GGTGCGGATC CCATCTGGTC GAAGCCCTGT
ACCTGGTCTG TGGCGAGGAA GGATTCTTCA AGAGGGCTGT
CAGGCCTCTG TGGGTGAGGA TGGAAAAGAG ATCCCTGAAA
AAAGGCGCCG CTGCCCTGGG AATTGGCACC GACTCCGTCA
TTCTCATCGA GGGCAGAGGA TCCCTCCTGA CCTGTGGCGA
CGTGGAGGAA AACCCCGGAC CCGAAGCTCT GTACCTGGTG
TGTGGCGAAA GGGGCTTTTT CCTGTCCGTC TACCTGAAAA
CCAATGTCTTTCTGTTTCTG CCCGGGTAGG TCGAC

The protein sequence for this construct is below (218 aa: SEQ ID NO: 29).

(SEQ ID NO: 29)
MMDQARSAFSNLFGGEPLSYTRFSLARQVDGDNSHVEMKLAVDEEENADN
NTKANVTKPKRCSGSICYGTIAVIVFFLIGFMIGYLGYCKGVEPKTECER
LAGTESPVREEPGEDFPAPRQGSHLVEALYLVCGEEGFFKRAVRPLWVRM
EKRSLKKGAAALGIGTDSVILIEGRGSLLTCGDVEENPGPEALYLVCGER
GFFLSVYLKTNVFLFLPG

TABLE 3

| Sequence Name | Sequence Location |
|---|---|
| endosome-targeting sequence (TFR1-118) | 1-118 |
| Ins2B: 9-23 R22E mimotope | 123-137 |
| OhgA 1040-79 mimotope | 142-151 |
| GAD65286-300 native peptide | 156-170 |
| cleavage site (T2A) | 173-190 |
| Ins2B: 15-23 native peptide | 193-201 |
| IGRP206-214 native peptide | 206-214 |

### Exemplary Epitope ONA and Amino Acid Sequences and Cleavage Sequences:

Ins28: 9-23
(SEQ ID NO: 30; epitope underlined)
TGTGGTTCCCACCTGGTGGAGGCTCTCTACCTGGTGTGTGGGGAGCGTGG
CTTCTTC
(SEQ ID NO: 31; epitope underlined)
CGSHLVEALYLVCGERGFF
Ins28: 9-23 R22E
(SEQ ID NO: 32; epitope underlined)
TGTGGTTCCCACCTGGTGGAGGCTCTCTACCTGGTGTGTGGGGAGGAGGG
CTTCTTC
(SEQ ID NO: 33; epitope underlined)
CGSHLVEALYLVCGEEGFF
Ins28: 9-23 E21G/R22E
(SEQ ID NO: 34)
TGTGGTTCCCACCTGGTGGAGGCTCTCTACCTGGTGTGTGGGGGAGAGGG
CTTCTTC
(SEQ ID NO: 35; epitope underlined)
CGSHLVEALYLVCGGEGFF
ChgA WE14
(SEQ ID NO: 36; epitope underlined)
AAGCGATGGAGCAGGATGGACCAGCTGGCCAAAGAGCTGACAGCAGAGAA
GCGG
(SEQ ID NO: 37; epitope underlined)
KRWSRMDQLAKELTAEKR
ChgA 1040-79
(SEQ ID NO: 38; epitope underlined)
AAGCGAGCAGTTCGACCTCTATGGGTACGTATGGAAAAGCGG
(SEQ ID NO: 39; epitope underlined)
KRAVRPLWVRMEKR
GAD65286-300
(SEQ ID NO: 40; epitope underlined)
TCTCTCAAGAAGGGAGCTGCAGCCTTAGGGATTGGAACAGAGAGTGTGAT
TCTGATT
(SEQ ID NO: 41; epitope underlined)
SLKKGAAALGIGTDSVILI
GAD65536.543
(SEQ ID NO: 42)
AGAATGAGCCGCCTCTCAAAGGTGGGGCCAGTGATTAAAGCCAGAATGAT
GGAGTATGGGACCACAATGGTC
(SEQ ID NO: 43; epitope underlined)
RMSRLSKVAPVIKARMMEYGTTMV
P2A (from porcine teschovirus-1)
(SEQ ID NO: 44)
GCTACTAACTTCAGCCTGCTGAAGCAGGCTGGAGACGTGGAGGAGAACCC
TGGACCT
(SEQ ID NO: 45)
ATNFSLLKQAGDVEENPGP
T2A (from Thosea asigna virus)
(SEQ ID NO: 46)
GAGGGCAGAGGAAGTCTGCTAACATGCGGTGACGTCGAGGAGAATCCTGG
ACCT
(SEQ ID NO: 47)
EGRGSLLTCGDVEENPGP
E2A (from equine rhinitis A virus)
(SEQ ID NO: 48)
CAGTGTACTAATTATGCTCTCTTGAAATTGGCTGGAGATGTTGAGAGCAA
CCCTGGACCT
(SEQ ID NO: 49)
QCTNYALLKLAGDVESNPGP
(SEQ ID NO: 50)
F2A (from Foot-and-mouth disease virus)
GTGAAACAGACTTTGAATTTTGACCTTCTCAAGTTGGGGGGAGACGTGGA
GTCCAACCCTGGACCT
(SEQ ID NO: 51)
VKQTLNFDLLKLAGDVESNPGP
Ins28: 15-23
(SEQ ID NO: 52; epitope underlined)
GAGGCTCTCTACCTGGTGTGTGGGGAGCGTGGCTTCTTC
(SEQ ID NO: 53; epitope underlined)
EALYLVCGERGFF
IGRP 200214
(SEQ ID NO: 54; epitope underlined)
TTGAGTGTGTACCTGAAGACCAACGTCTTCCTCTTCCTG
(SEQ ID NO: 55; epitope underlined)
LSVYLKTNVFLFL
GAD65₂₀₆₋₂₁₄
(SEQ ID NO: 56)
TYEIAPVFV
GAD65₅₄₆₋₅₅₄
(SEQ ID NO: 57)
SYQPLGDKV

### STAT1c

Proteins in accordance with the disclosure may be produced by changing (that is, modifying) a wild-type protein to produce a new protein with a novel combination of useful protein characteristics, such as altered Vmax, Km, substrate specificity, substrate selectivity, and protein stability. Modifications may be made at specific amino acid positions in a protein and may be a substitution of the amino acid found at that position in nature (that is, in the wild-type protein) with a different amino acid. Proteins provided by the disclosure thus provide a new protein with one or more altered protein characteristics relative to the wild-type protein found in nature. In one embodiment of the disclosure, a protein may have altered protein characteristics such as improved or decreased activity against one or more herbicides or improved protein stability as compared to a similar wild-type protein, or any combination of such characteristics. In one embodiment, the disclosure provides a protein, and the DNA molecule or coding sequence encoding it, having at least about 80% sequence identity, about 81% sequence identity, about 82% sequence identity, about 83% sequence identity, about 84% sequence identity, about 85% sequence identity, about 86% sequence identity, about 87% sequence identity, about 88% sequence identity, about 89% sequence identity, about 90% sequence identity, about 91% sequence identity, about 92% sequence identity, about 93% sequence identity, about 94% sequence identity, about 95% sequence identity, about 96% sequence identity, about 97% sequence identity, about 98% sequence identity, about 99% sequence identity, or about 100% sequence identity to a protein sequence such as set forth as SEQ ID NOs: 3 and 4. Amino acid mutations may be made as a single amino acid substitution in the protein or in combination with one or more other mutation(s), such as one or more other amino acid substitution(s), deletions, or additions. Mutations may be made as described herein or by any other method known to those of skill in the art.

### Stat1 Sequences:

STAT1 protein alignments (from CDS sequences):
93% identity between mouse and human
Mm: mouse sequence (Mus musculus)
Hs: human sequence (Homo sapiens)
LF: long form (splice isoform)
SF: short form (splice isoform)
SC: STAT1c (constitutively active mutant)

Highlighted in bold and underlined are the mutated sites to render the protein constitutively active (Refs: Sironi & Ouchi, 2004, JBC, STAT1-induced Apoptosis Is Mediated by Caspases 2, 3, and 7.

Liddle, Alvarez, Poli and Frank, 2006, Biochem., Tyrosine Phosphorylation Is Required for Functional Activation of Disulfide-Containing Constitutively Active STAT Mutants). The descending order of SEQ ID NOs for the below table is SEQ ID NO: 7, SEQ ID NO:8, SEQ ID NO:4, SEQ ID NO:5, SEQ ID NO:6, SEQ ID NO:3.

TABLE 4

| | |
|---|---|
| Mm_Stat1_LF | MSQWFELQQLDSKFLEQVHQLYDDSFPMEIRQYLAQWLEKQDWEHAAYDVSFATIRFHDLLSQLDDQYSRFS |
| Mm_Stat1_SF | MSQWFELQQLDSKFLEQVHQLYDDSFPMEIRQYLAQWLEKQDWEHAAYDVSFATIRFHDLLSQLDDQYSRFS |
| Mm_Stat1_SC | MSQWFELQQLDSKFLEQVHQLYDDSFPMEIRQYLAQWLEKQDWEHAAYDVSFATIRFHDLLSQLDDQYSRFS |
| Hs_Stat1_LF | MSQWFELQQLDSKFLEQVHQLYDDSFPMEIRQYLAQWLEKQDWEHAANDVSFATIRFHDLLSQLDDQYSRFS |
| Hs_Stat1_SF | MSQWYELQQLDSKFLEQVHQLYDDSFPMEIRQYLAQWLEKQDWEHAANDVSFATIRFHDLLSQLDDQYSRFS |
| Hs_Stat1_SC | MSQWFELQQLDSKFLEQVHQLYDDSFPMEIRQYLAQWLEKQDWEHAANDVSFATIRFHDLLSQLDDQYSRFS |
| Mm_Stat1_LF | LENNFLLQHNIRKSKRNLQDNFQEDPVQMSMIIYNCLKEERKILENAQRFNQAQEGNIQNTVMLDKQKELDS |
| Mm_Stat1_SF | LENNFLLQHNIRKSKRNLQDNFQEDPVQMSMIIYNCLKEERKILENAQRFNQAQEGNIQNTVMLDKQ3KELDS |
| Mm_Stat1_SC | LENNFLLQHNIRKSKRNLQDNFQEDPVQMSMIIYNCLKEERKILENAQRFNQAQEGNIQNTVMLDKQKELDS |
| Hs_Stat1_LF | LENNFLLQHNIRKSKRNLQDNFQEDPIQMSMIIYSCLKEERKILENAQRFNQAQSGNIQSTVMLDKQKELDS |
| Hs_Stat1_SF | LENNFLLQHNIRKSKRNLQDNFQEDPIQMSMIIYSCLKEERKILENAQRFNQAQSGNIQSTVMLDKQKELDS |
| Hs_Stat1_SC | LENNFLLQHNIRKSKRNLQDNFQEDPIQMSMIIYSCLKEERKILENAQRFNQAQSGNIQSTVMLDKQKELDS |
| Mm_Stat1_LF | KVRNVKDQVMCIEQEIKTLEELQDEYDFKCKTSQNREGEANGVAKSDQKQEQLLLHKMFLMLDNKRKEIIHK |
| Mm_Stat1_SF | KVRNVKDQVMCIEQEIKTLEELQDEYDFKCKTSQNREGEANGVAKSDQKQEQLLLHKMFLMLDNKRKEIIHK |
| Mm_Stat1_SC | KVRNVKDQVMCIEQEIKTLEELQDEYDFKCKTSQNREGEANGVAKSDQKQEQLLLHKMFLMLDNKRKEIIHK |
| Hs_Stat1_LF | KVRNVKDKVMCIEHEIKSLEDLQDEYDFKCKTLQNREHETNGVAKSDQKQEQLLLKKMYLMLDNKRKEVVHK |
| Hs_Stat1_SF | KVRNVKDKVMCIEHEIKSLEDLQDEYDFKCKTLQNREHETNGVAKSDQKQEQLLLKKMYLMLDNKRKEVVHK |
| Hs_Stat1_SC | KVRNVKDKVMCIEHEIKSLEDLQDEYDFKCKTLQNREHETNGVAKSDQKQEQLLLKKMYLMLDNKRKEVVHK |
| Mm_Stat1_LF | IRELLNSIELTQNTLINDELVEWKRRQQSACIGGPPNACLDQLQSWFTIVAETLQQIRQQLKKLEELEQKFT |
| Mm_Stat1_SF | IRELLNSIELTQNTLINDELVEWKRRQQSACIGGPPNACLDQLQSWFTIVAETLQQIRQQLKKLEELEQKFT |
| Mm_Stat1_SC | IRELLNSIELTQNTLINDELVEWKRRQQSACIGGPPNACLDQLQSWFTIVAETLQQIRQQLKKLEELEQKFT |
| Hs_Stat1_LF | IIELLNVTELTQNALINDELVEWKRRQQSACIGGPPNACLDQLQNWFTIVAESLQQVRQQLKKLEELEQKYT |
| Hs_Stat1_SF | IIELLNVTELTQNALINDELVEWKRRQQSACIGGPPNACLDQLQNWFTIVAESLQQVRQQLKKLEELEQKYT |
| Hs_Stat1_SC | IIELLNVTELTQNALINDELVEWKRRQQSACIGGPPNACLDQLQNWFTIVAESLQQVRQQLKKLEELEQKYT |
| Mm_Stat1_LF | YEPDPITKNKQVLSDRTFLLFQQLIQSSFVVERQPCMPTHPQRPLVLKTGVQFTVKLRLLVKLQELNYNLKV |
| Mm_Stat1_SF | YEPDPITKNKQVLSDRTFLLFQQLIQSSFVVERQPCMPTHPQRPLVLKTGVQFTVKLRLLVKLQELNYNLKV |
| Mm_Stat1_SC | YEPDPITKNKQVLSDRTFLLFQQLIQSSFVVERQPCMPTHPQRPLVLKTGVQFTVKLRLLVKLQELNYNLKV |
| Hs_Stat1_LF | YEHDPITKNKQVLWDRTFSLFQQLIQSSFVVERQPCMPTHPQRPLVLKTGVQFTVKLRLLVKLQELNYNLKV |
| Hs_Stat1_SF | YEHDPITKNKQVLWDRTFSLFQQLIQSSFVVERQPCMPTHPQRPLVLKTGVQFTVKLRLLVKLQELNYNLKV |
| Hs_Stat1_SC | YEHDPITKNKQVLWDRTFSLFQQLIQSSFVVERQPCMPTHPQRPLVLKTGVQFTVKLRLLVKLQELNYNLKV |
| Mm_Stat1_LF | KVSFDKDVNEKNTVKGFRKFNILGTHTKVMNMEESTNGSLAAEFRHLQLKEQKNAGNRTNEGPLIVTEELHS |
| Mm_Stat1_SF | KVSFDKDVNEKNTVKGFRKFNILGTHTKVMNMEESTNGSLAAEFRHLQLKEQKNAGNRTNEGPLIVTEELHS |
| Mm_Stat1_SC | KVSFDKDVNEKNTVKGFRKFNILGTHTKVMNMEESTNGSLAAEFRHLQLKEQKNAGNRTNEGPLIVTEELHS |
| Hs_Stat1_LF | KVLFDKDVNERNTVKGFRKFNILGTHTKVMNMEESTNGSLAAEFRHLQLKEQKNAGTRTNEGPLIVTEELHS |
| Hs_Stat1_SF | KVLFDKDVNERNTVKGFRKFNILGTHTKVMNMEESTNGSLAAEFRHLQLKEQKNAGTRTNEGPLIVTEELHS |
| Hs_Stat1_SC | KVLFDKDVNERNTVKGFRKFNILGTHTKVMNMEESTNGSLAAEFRHLQLKEQKNAGTRTNEGPLIVTEELHS |
| Mm_Stat1_LF | LSFETQLCQPGLVIDLEVFVPFQTTSLPVVVISNVSQLPSGWASILWYNMLVTEPRNLSFFLNPPCAWWSQL |
| Mm_Stat1_SF | LSFETQLCQPGLVIDLE------TTSLPVVVISNVSQLPSGWASILWYNMLVTEPRNLSFFLNPPCAWWSQL |
| Mm_Stat1_SC | LSFETQLCQPGLVIDLE------TTSLPVVVISNVSQLPSGWASILWYNMLVTEPRNLSFFLNPPCAWWSQL |
| Hs_Stat1_LF | LSFETQLCQPGLVIDLE------TTSLPVVVISNVSQLPSGWASILWYNMLVAEPRNLSFFLTPPCARWAQL |
| Hs_Stat1_SF | LSFETQLCQPGLVIDLE------TTSLPVVVISNVSQLPSGWASILWYNMLVAEPRNLSFFLTPPCARWAQL |
| Hs_Stat1_SC | LSFETQLCQPGLVIDLE------TTSLPVVVISNVSQLPSGWASILWYNMLVAEPRNLSFFLTPPCARWAQL |
| Mm_Stat1_LF | SEVLSWQFSSVTKRGLNADQLSMLGEKLLGPNAGPDGLIPWTRFCKENINDKNFSFWPWIDTILELIKKHLL |
| Mm_Stat1_SF | SEVLSWQFSSVTKRGLNADQLSMLGEKLLGPNAGPDGLIPWTRFCKENINDKNFSFWPWIDTILELIKKHLL |
| Mm_Stat1_SC | SEVLSWQFSSVTKRGLNADQLSMLGEKLLGPNAGPDGLIPWTRFCKENINDKNFSFWPWIDTILELIKKHLL |
| Hs_Stat1_LF | SEVLSWQFSSVTKRGLNVDQLNMLGEKLLGPNASPDGLIPWTRFCKENINDKNFPFWLWIESILELIKKHLL |
| Hs_Stat1_SF | SEVLSWQFSSVTKRGLNVDQLNMLGEKLLGPNASPDGLIPWTRFCKENINDKNFPFWLWIESILELIKKHLL |
| Hs_Stat1_SC | SEVLSWQFSSVTKRGLNVDQLNMLGEKLLGPNASPDGLIPWTRFCKENINDKNFPFWLWIESILELIKKHLL |
| Mm_Stat1_LF | CLWNDGCIMGFISKERERALLKDQQPGTFLLRFSESSREGAITFTWVERSQNGGEPDFHAVEPYTKKELSAV |
| Mm_Stat1_SF | CLWNDGCIMGFISKERERALLKDQQPGTFLLRFSESSREGAITFTWVERSQNGGEPDFHAVEPYTKKELSAV |
| Mm_Stat1_SC | CLWNDGCIMGFISKERERALLKDQQPGTFLLRFSESSREGAITFTWVERSQNGGEPDFHAVEPYTKKELSAV |
| Hs_Stat1_LF | PLWNDGCIMGFISKERERALLKDQQPGTFLLRFSESSREGAITFTWVERSQNGGEPDFHAVEPYTKKELSAV |
| Hs_Stat1_SF | PLWNDGCIMGFISKERERALLKDQQPGTFLLRFSESSREGAITFTWVERSQNGGEPDFHAVEPYTKKELSAV |
| Hs_Stat1_SC | PLWNDGCIMGFISKERERALLKDQQPGTFLLRFSESSREGAITFTWVERSQNGGEPDFHAVEPYTKKELSAV |
| Mm_Stat1_LF | TFPDIIRNYKVMAAENIPENPLKYLYPNIDKDHAFGKYYSRPKEAPEPMELDDPKRTGYIKTELISVSEVHP |
| Mm_Stat1_SF | TFPDIIRNYKVMAAENIPENPLKYLYPNIDKDHAFGKYYSRPKEAPEPMELDDPKRTGYIKTELISVSEVHP |
| Mm_Stat1_SC | TFPDIIRNYKVMAAENIPENPLKYLYPNIDKDHAFGKYYSRPKEAPEPMELDDPKRTGYIKTELISVSEVHP |
| Hs_Stat1_LF | TFPDIIRNYKVMAAENIPENPLKYLYPNIDKDHAFGKYYSRPKEAPEPMELDGPKGTGYIKTELISVSEVHP |
| Hs_Stat1_SF | TFPDIIRNYKVMAAENIPENPLKYLYPNIDKDHAFGKYYSRPKEAPEPMELDGPKGTGYIKTELISVSEVHP |
| Hs_Stat1_SC | TFPDIIRNYKVMAAENIPENPLKYLYPNIDKDHAFGKYYSRPKEAPEPMELDGPKGTGYIKTELISVSEVHP |
| Mm_Stat1_LF | SRLQTTDNLLPMSPEEFDEMSRIVGP-EFDSMMSTV |
| Mm_Stat1_SF | SRLQTTDNLLPMSPEEFDEMSRIVGP-EFDSMMSTV |
| Mm_Stat1_SC | SRLQTTDNLLPMSPEEFDEMSRIVGP-EFDSMMSTV |
| Hs_Stat1_LF | SRLQTTDNLLPMSPEEFDEVSRIVGSVEFDSMMNTV |
| Hs_Stat1_SF | |
| Hs_Stat1_SC | SRLQTTDNLLPMSPEEFDEVSRIVGSVEFDSMMNTV |

Human STAT1c DNA sequence:
SEQ ID NO: 1
ATGTCTCAGTGGTACGAACTTCAGCAGCTTGACTCAAAATTCCTGGAGCAGGTTCACCAGCTTTATGATGACAGTTT
TCCCATGGAAATCAGACAGTACCTGGCACAGTGGTTAGAAAAGCAAGACTGGGAGCACGCTGCCAATGATGTTTCAT
TTGCCACCATCCGTTTTCATGACCTCCTGTCACAGCTGGATGATCAATATAGTCGCTTTTCTTTGGAGAATAACTTC
TTGCTACAGCATAACATAAGGAAAAGCAAGCGTAATCTTCAGGATAATTTTCAGGAAGACCCAATCCAGATGTCTAT
GATCATTTACAGCTGTCTGAAGGAAGAAAGGAAAATTCTGGAAAACGCCCAGAGATTTAATCAGGCTCAGTCGGGGA
ATATTCAGAGCACAGTGATGTTAGACAAACAGAAAGAGCTTGACAGTAAAGTCAGAAATGTGAAGGACAAGGTTATG
TGTATAGAGCATGAAATCAAGAGCCTGGAAGATTTACAAGATGAATATGACTTCAAATGCAAAACCTTGCAGAACAG
AGAACACGAGACCAATGGTGTGGCAAAGAGTGATCAGAAACAAGAACAGCTGTTACTCAAGAAGATGTATTTAATGC
TTGACAATAAGAGAAAGGAAGTAGTTCACAAAATAATAGAGTTGCTGAATGTCACTGAACTTACCCAGAATGCCCTG
ATTAATGATGAACTAGTGGAGTGGAAGCGGAGACAGCAGAGCGCCTGTATTGGGGGGCCGCCCAATGCTTGCTTGGA
TCAGCTGCAGAACTGGTTCACTATAGTTGCGGAGAGTCTGCAGCAAGTTCGGCAGCAGCTTAAAAAGTTGGAGGAAT
TGGAACAGAAATACACCTACGAACATGACCCTATCACAAAAAACAAACAAGTGTTATGGGACCGCACCTTCAGTCTT
TTCCAGCAGCTCATTCAGAGCTCGTTTGTGGTGGAAAGACAGCCCTGCATGCCAACGCACCCTCAGAGGCCGCTGGT
CTTGAAGACAGGGGTCCAGTTCACTGTGAAGTTGAGACTGTTGGTGAAATTGCAAGAGCTGAATTATAATTTGAAAG
TCAAAGTCTTATTTGATAAAGATGTGAATGAGAGAAATACAGTAAAAGGATTTAGGAAGTTCAACATTTTGGGCACG
CACACAAAAGTGATGAACATGGAGGAGTCCACCAATGGCAGTCTGGCGGCTGAATTTCGGCACCTGCAATTGAAAGA
ACAGAAAAATGCTGGCACCAGAACGAATGAGGGTCCTCTCATCGTTACTGAAGAGCTTCACTCCCTTAGTTTTGAAA
CCCAATTGTGCCAGCCTGGTTTGGTAATTGACCTCGAGACGACCTCTCTGCCCGTTGTGGTGATCTCCAACGTCAGC
CAGCTCCCGAGCGGTTGGGCCTCCATCCTTTGGTACAACATGCTGGTGGCGGAACCCAGGAATCTGTCCTTCTTCCT
GACTCCACCATGTGCACGATGGGCTCAGCTTTCAGAAGTGCTGAGTTGGCAGTTTTCTTCTGTCACCAAAAGAGGTC
TCAATGTGGACCAGCTGAACATGTTGGGAGAGAAGCTTCTTGGTCCTAACGCCAGCCCCGATGGTCTCATTCCGTGG
ACGAGGTTTTGTAAGGAAAATATAAATGATAAAAATTTTCCCTTCTGGCTTTGGATTGAAAGCATCCTAGAACTCAT
TAAAAAACACCTGCTCCCTCTCTGGAATGATGGGTGCATCATGGGCTTCATCAGCAAGGAGCGAGAGCGTGCCCTGT
TGAAGGACCAGCAGCCGGGGACCTTCCTGCTGCGGTTCAGTGAGAGCTCCCGGGAAGGGGCCATCACATTCACATGG
GTGGAGCGGTCCCAGAACGGAGGCGAACCTGACTTCCATGCGGTTGAACCCTACACGAAGAAAGAACTTTCTGCTGT
TACTTTCCCTGACATCATTCGCAATTACAAAGTCATGGCTTGTGAGTGTATTCCTGAGAATCCCCTGAAGTATCTGT
ATCCAAATATTGACAAAGACCATGCCTTTGGAAAGTATTACTCCAGGCCAAAGGAAGCACCAGAGCCAATGGAACTT
GATGGCCCTAAAGGAACTGGATATATCAAGACTGAGTTGATTTCTGTGTCTGAAGTTCACCCTTCTAGACTTCAGAC
CACAGACAACCTGCTCCCCATGTCTCCTGAGGAGTTTGACGAGGTGTCTCGGATAGTGGGCTCTGTAGAATTCGACA
GTATGATGAACACAGTATAG
Mouse STAT1c DNA sequence:
SEQ ID NO: 2
ATGTCACAGTGGTTCGAGCTTCAGCAGCTGGACTCCAAGTTCCTGGAGCAGGTCCACCAGCTGTACGATGACAGTTT
CCCCATGGAAATCAGACAGTACCTGGCCCAGTGGCTGGAAAAGCAAGACTGGGAGCACGCTGCCTATGATGTCTCGT
TTGCGACCATCCGCTTCCATGACCTCCTCTCACAGCTGGACGACCAGTACAGCCGCTTTTCTCTGGAGAATAATTTC
TTGTTGCAGCACAACATACGGAAAAGCAAGCGTAATCTCCAGGATAACTTCCAAGAAGATCCCGTACAGATGTCCAT
GATCATCTACAACTGTCTGAAGGAAGAAAGGAAGATTTTGGAAAATGCCCAAAGATTTAATCAGGCCCAGGAGGGAA
ATATTCAGAACACTGTGATGTTAGATAAACAGAAGGAGCTGGACAGTAAAGTCAGAAATGTGAAGGATCAAGTCATG
TGCATAGAGCAGGAAATCAAGACCCTAGAAGAATTACAAGATGAATATGACTTTAAATGCAAAACCTCTCAGAACAG
AGAAGGTGAAGCCAATGGTGTGGCGAAGAGCGACCAAAAACAGGAACAGCTGCTGCTCCACAAGATGTTTTTAATGC
TTGACAATAAGAGAAAGGAGATAATTCACAAAATCAGAGAGTTGCTGAATTCCATCGAGCTCACTCAGAACACTCTG
ATTAATGACGAGCTCGTGGAGTGGAAGCGAAGGCAGCAGAGCGCCTGCATCGGGGGACCGCCCAACGCCTGCCTGGA
TCAGCTGCAAAGCTGGTTCACCATTGTTGCAGAGACCCTGCAGCAGATCCGTCAGCAGCTTAAAAAGCTGGAGGAGT
TGGAACAGAAATTCACCTATGAGCCCGACCCTATTACAAAAAACAAGCAGGTGTTGTCAGATCGAACCTTCCTCCTC
TTCCAGCAGCTCATTCAGAGCTCCTTCGTGGTAGAACGACAGCCGTGCATGCCCACTCACCCGCAGAGGCCCCTGGT
CTTGAAGACTGGGGTACAGTTCACTGTCAAGCTGAGACTGTTGGTGAAATTGCAAGAGCTGAACTATAACTTGAAAG
TGAAAGTCTCATTTGACAAAGATGTGAACGAGAAAAACACAGTTAAAGGATTTCGGAAGTTCAACATCTTGGGTACG
CACACAAAAGTGATGAACATGGAAGAATCCACCAACGGAAGTCTGGCAGCTGAGTTCCGACACCTGCAACTGAAGGA
ACAGAAAAACGCTGGGAACAGAACTAATGAGGGGCCTCTCATTGTCACCGAAGAACTTCACTCTCTTAGCTTTGAAA
CCCAGTTGTGCCAGCCAGGCTTGGTGATTGACCTGGAGGTCTTTGTTCCCTTTCAGACCACCTCTCTTCCTGTCGTG
GTGATCTCCAACGTCAGCCAGCTCCCCAGTGGCTGGGCGTCTATCCTGTGGTACAACATGCTGGTGACAGAGCCCAG
GAATCTCTCCTTCTTCCTGAACCCCCCGTGCGCGTGGTGGTCCCAGCTCTCAGAGGTGTTGAGTTGGCAGTTTTCAT
CAGTCACCAAGAGAGGTCTGAACGCAGACCAGCTGAGCATGCTGGGAGAGAAGCTGCTGGGCCCTAATGCTGGCCCT
GATGGTCTTATTCCATGGACAAGGTTTTGTAAGGAAAATATTAATGATAAAAATTTCTCCTTCTGGCCTTGGATTGA
CACCATCCTAGAGCTCATTAAGAAGCACCTGCTGTGCCTCTGGAATGATGGGTGCATTATGGGCTTCATCAGCAAGG
AGCGAGAACGCGCTCTGCTCAAGGACCAGCAGCCAGGGACGTTCCTGCTTAGATTCAGTGAGAGCTCCCGGGAAGGG
GCCATCACATTCACATGGGTGGAACGGTCCCAGAACGGAGGTGAACCTGACTTCCATGCCGTGGAGCCCTACACGAA
AAAAGAACTTTCAGCTGTTACTTTCCCAGATATTATTCGCAACTACAAAGTCATGGCTTGCGAGTGCATACCAGAGA
ATCCCCTGAAGTATCTGTACCCCAATATTGACAAAGACCACGCCTTTGGGAAGTATTATTCCAGACCAAAGGAAGCA
CCAGAACCGATGGAGCTTGACGACCCTAAGCGAACTGGATACATCAAGACTGAGTTGATTTCTGTGTCTGAAGTCCA
CCCTTCTAGACTTCAGACCACAGACAACCTGCTTCCCATGTCTCCAGAGGAGTTTGATGAGATGTCCCGGATAGTGG
GCCCCGAATTTGACAGTATGATGAGCACAGTATAA

miRNA Targeting

In some cases, a modification is conducted at a target sequence, or at a target sequence that is at least 95 percent (e.g., at least 96 percent, at least 97 percent, at least 98 percent, or at least 99 percent) identical to the target sequence. In a more specific example, a modification is conducted at a target sequence set forth in SEQ ID NOs: 9 or 10, or at a target sequence that is at least 95 percent (e.g., at least 96 percent, at least 97 percent, at least 98 percent, or at least 99 percent) identical to a sequence set forth in SEQ ID Nos 9 or 10.

### Mir-142-T Sequences:

miR-142Tsingle site:
(SEQ ID NO: 9)
TCCATAAAGTAGGAAACACTACA
miR-142T4X site
(for efficient targeting by miR-142):
(SEQ ID NO: 10)
CTAGAGTCGACTCCATAAAGTAGGAAACACTACACGATTCCATAAAGTA
GGAAACACTACAACCGGTTCCATAAAGTAGGAAACACTACATCACTCCA
TAAAGTAGGAAACACTACAC

### Refs:

Brown, B.D., Venneri, M. A., Zingale, A., Sergi Sergi, L. & Naldini, L. Endogenous microRNA regulation suppresses transgene expression in hematopoietic lineages and enables stable gene transfer. Nat Med 12, 585-591 (2006).

Cire, S., Da Rocha, S., Ferrand, M., Collins, M. K. & Galy, A. In Vivo Gene Delivery to Lymph Node Stromal Cells Leads to Transgene-specific CD8+ T Cell Anergy in Mice. Mol Ther (2016).

### Sequence Identity and Modification

The percent sequence identity between a particular nucleic acid or amino acid sequence and a sequence referenced by a particular sequence identification number may be determined by techniques known in the art. In one example, sequence identity is determined as follows. First, a nucleic acid or amino acid sequence is compared to the sequence set forth in a particular sequence identification number using the BLAST 2 Sequences (B12seq) program from the stand-alone version of BLASTZ containing BLASTN version 2.0.14 and BLASTP version 2.0.14. This stand-alone version of BLASTZ can be obtained online at fr.com/blast or at ncbi.nlm.nih.gov. Instructions explaining how to use the B12 seq program can be found in the readme file accompanying BLASTZ. B12seq performs a comparison between two sequences using either the BLASTN or BLASTP algorithm. BLASTN is used to compare nucleic acid sequences, while BLASTP is used to compare amino acid sequences. To compare two nucleic acid sequences. the options are set as follows: −i is set to a file containing the first nucleic acid sequence to be compared (e.g., C:\seq1.txt); −j is set to a file containing the second nucleic acid sequence to be compared (e.g., C:\seq2.txt); −p is set to blastn; −o is set to any desired file name (e.g., C:\output.txt); −q is set to −1; −r is set to 2; and all other options are left at their default setting. For example, the following command can be used to generate an output file containing a comparison between two sequences: C: \B12seq c:\seq1.txt-j c:\seq2.txt-p blastn-o c:\output.txt-q -1-r 2. To compare two amino acid sequences, the options of B12seq are set as follows: −i is set to a file containing the first amino acid sequence to be compared (e.g., C:\seq1.txt); −j is set to a file containing the second amino acid sequence to be compared (e.g., C:\seq2.txt); −p is set to blastp; −o is set to any desired file name (e.g., C:\output.txt); and all other options are left at their default setting. For example, the following command can be used to generate an output file containing a comparison between two amino acid sequences: C:\B12seq c:\seq2.txt-j c:\seq2.txt-p blastp-o c:\output.txt. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (e.g., 100 consecutive nucleotides or amino acid residues from a sequence set forth in an identified sequence), followed by multiplying the resulting value by 100. For example, a nucleic acid sequence that has x matches when aligned with a first sequence is x percent identical to the sequence set forth in second sequence (i.e., x+y×100=%). It is noted that the percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. It also is noted that the length value will always be an integer.

### Secretion Signal

Certain embodiments of the nucleic acid constructs may implement a secretion signal sequence for secreting the one or epitopes from the transfected cell. An example of a secretion signal sequence pertains to a codon-optimized albumin secretion signal:

(SEQ ID NO: 24)
ATGAAGTGGGTAACCTTTCTCCTCCTCCTCTTCATCTCCGGTTCTGCCTT
TTCTAGGGGCAAGCTTATG

Those skilled in the art will appreciate that other known secretion signal sequences may be implemented in the nucleic acid constructs. A secretory signal sequence can be obtained from other eukaryotic polypeptides that are known to be secreted. With the cloning and sequencing of numerous genomes, including human, there exists a wide variety of eukaryotic secretion signal sequences that can be employed. Ideally, the secretion signal sequence is selected from a species from transfection of cells is intended, or codon-optimized for that species. In addition to the codon-optimized albumin secretion signal provided above, other examples include an albumin leader having the sequence ATG AAG TGG GTA ACC TTT ATT TCC CTT CTT TTT CTC TTT AGC TCG GCT TAT TCC AGG GGT GTG TTT CGT CGA GAT (SEQ ID NO: 25) and an immunoglobulin kappa (Ig K)-chain leader having the sequence ATG GAG ACA GAC ACA CTC CTG CTA TGG GTA CTG CTG CTC TGG GTT CCA GGT TCC ACT GGT GAC (SEQ ID NO: 26). See also U.S. Pat. No. 9,157,085 and WO/2014/177826 for other examples that may be adapted for use with nucleic acid construct embodiments.

### Nanoparticle Formulation

The nucleic acid constructs described herein may also be complexed with polycationic molecules (including proteins, lipids, and polymers thereof) or liposomes that enhance cell transfection. However, such complexes tend to be rapidly degraded in professional APCs such as DCs, thus productive transfection tends to be more successful in stromal cells. As explained above, presentation of dual CD4 and CD8 epitopes by stromal cells, or secretion of these epitopes by stromal cells increases the likelihood of inducing a tolerogenic response to such epitopes. Examples of polycationic molecules include, but are not limited to, positively charged cell-penetrating peptides (CPPs, such as polyarginine, polylysine or HIV Tat peptide), used in conjunction with calcium or not, or positively charged polymer molecules (such as polyethylenimine) and cationic lipids. The polycationic molecules associate with negative charges on the nucleic acid construct so as to fold and condense the construct. This condensing makes the construct smaller, which in turn facilitates migration of the construct and easier uptake by cells.

The nucleic acid constructs disclosed herein may be associated with polycationic molecules that serve to enhance uptake by cells. Complexing the nucleic acid construct with polycationic molecules also helps in packaging the construct such their size is reduced, which is believed to assist with cellular uptake and in vivo dispersion, including improved delivery to lymph nodes to target LNSCs. Once in the endosome, the complex dissociates due to the lower pH, and the polycationic molecules can disrupt the endosome's membrane to facilitate DNA escape into the cytoplasm before it can be degraded. Published data show that the nucleic acid construct embodiments had enhanced uptake into SCs over DCs when complexed with cationic lipids (21).

One example of polycationic molecules useful for complexing with nucleic acid constructs includes CPPs, examples include polylysine (described above), polyarginine and Tat peptides. CPPs are small peptides which can bind to DNA and, once released, penetrate cell membranes to facilitate escape of the DNA/mRNA from the endosome to the cytoplasm. Another example of a CPP pertains to a 27-residue chimeric peptide, termed MPG, was shown some time ago to bind ss- and ds-oligonucleotides in a stable manner, resulting in a non-covalent complex that protected the nucleic acids from degradation by DNase and effectively delivered oligonucleotides to cells in vitro (Mahapatro A, et al., J Nanobiotechnol, 2011, 9:55). The complex formed small particles of approximately 150 nm to 1 um when different peptide:DNA ratios were examined, and the 10:1 and 5:1 ratios (150 nm and 1 um respectively). Another CPP pertains to a modified tetrapeptide [tetralysine containing guanidinocarbonylpyrrole (GCP) groups (TL-GCP)], which was reported to bind with high affinity to a 6.2 kb plasmid DNA resulting in a positive charged aggregate of 700-900 nm Li et al., Agnew Chem Int Ed Enl 2015; 54(10):2941-4). RNA can also be complexed by such polycationic molecules for in vivo delivery (see review by Yin & Anderson).

Other examples of polycationic molecules that may be complexed with the nucleic acid constructs described herein include polycationic polymers commercially available as JETPRIME® and in vivo-jetPEI® (with polyethylenimine) and in vivo-jetRNA® (with cationic lipids) (Polypus-transfection, S. A., Illkirch, France).

### Compositions, Administration, Routes, and Doses of Vaccines

The nucleic constructs disclosed herein are contemplated for administration to a subject in need, and can be administered by any convenient method known to the person of skill in the art. Administration can be by any route, including but not limited to local and systemic methods, for example aerosols for delivery to the lung, oral, rectal, vaginal, buccal, transmucosal, intranodal, transdermal, subcutaneous, intravenous, subcutaneous, intradermal, intratracheal, intramuscular, intraarterial, intraperitoneal, intracranial (e.g., intrathecal or intraventricular) or any known and convenient route. Preferred routes of administration are intravenous, intraperitoneal, subcutaneous, oral/nasal and direct injection into the affected organ, tissue, arca of infection or tumor, or specific lymph nodes. The form of the administration can determine how the active agent is formulated, and this is easily determined by the skilled artisan. Nucleic acid drugs generally are delivered in nanosized drug formulations into the blood stream, and these well-known formulations and methods of administration are preferred. An exemplary nanocarrier is described in Pujol-Autonell et al., “Use of autoantigen-loaded phosphatidylserine-liposomes to arrest autoimmunity in type 1 diabetes.” PloS one 10, e0127057 (2015).

Compositions embodiments comprising one or nucleic acid constructs therefore can include, but are not limited to, solid preparations for oral administration, solid preparations to be dissolved in a liquid carrier for oral or parenteral administration, solutions, suspensions, emulsions, oils, creams, ointments, lotions, gels, powders, granules, cells in suspension, and liposome-containing formulations, and the like, or any convenient form known in the art. These compositions may be generated from a variety of components that include, but are not limited to, preformed liquids, self-emulsifying solids and self-emulsifying semisolids.

Solutions or suspensions used for parenteral, intradermal, subcutaneous or other injection can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylene diamine tetra acetic acid; buffers such as acetates, citrates or phosphates; and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Nucleic Acid construct containing compositions suitable for injectable use include sterile aqueous solutions (where the therapeutic agents are water soluble) or dispersions and sterile powders for the extemporaneous preparation of sterile injectable solutions or dispersion. For intravenous administration, suitable carriers include physiological saline, bacteriostatic water, Cremophor EL® (BASF, Parsippany, N.J.) or phosphate buffered saline (PBS). In all cases, the composition must be sterile and should be fluid to the extent that they can pass through a syringe and needle easily enough for administration. It should be stable under the conditions of manufacture and storage and should be preserved against the contaminating action of microorganisms such as bacteria and fungi. All solutions used to solubilize DNA or RNA should also be DNase-free and RNase-frec.

The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions comprising one or more disclosed nucleic acid constructs can be prepared by incorporating the active agent in the required amount in an appropriate solvent with one or a combination of the ingredients enumerated above, as required, followed by filter sterilization. Generally, dispersions are prepared by incorporating the active agent into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof. The skilled person is aware of how to use these dried preparations for injection.

Oral compositions comprising one or more disclosed nucleic acid constructs generally include an inert diluent or an edible carrier. They can be enclosed in gelatin capsules or compressed into tablets. Depending on the specific conditions being treated, pharmaceutical compositions of the present invention for treatment of atherosclerosis or the other elements of metabolic syndrome can be formulated and administered systemically or locally. Techniques for formulation and administration can be found in “Remington: The Science and Practice of Pharmacy” (20th edition, Gennaro (ed.) and Gennaro, Lippincott, Williams & Wilkins, 2000). For oral administration, the agent can be contained in enteric forms to survive the stomach or further coated or mixed to be released in a particular region of the GI tract by known methods. For the purpose of oral therapeutic administration, the active agent can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, PRIMOGEL® or corn starch; a lubricant such as magnesium stearate or STEROTES®; a glidant such as colloidal silicon dioxide; a sweetening agent such as sucrose or saccharin; or a flavoring agent such as peppermint, methyl salicylate, or orange flavoring.

Systemic administration can also be by transmucosal means to the intestinal or colon, such as by suppository or enema, for example. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the disclosed nucleic acid constructs are formulated into ointments, salves, gels, or creams as generally known in the art.

In several embodiments, the disclosed nucleic acid constructs are prepared with carriers that will protect the compound against rapid elimination from the body, such as a controlled release or delayed formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to particular cells with, e.g., monoclonal antibodies) can also be used as pharmaceutically acceptable carriers. These can be prepared according to methods known to those skilled in the art.

Formulations comprising one or more disclosed nucleic acid constructs designed to provide extended or delayed release also are contemplated for use with the invention. The following United States patents contain representative teachings concerning the preparation of uptake, distribution and/or absorption assisting formulations: U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756. Such compositions are contemplated for use with the invention.

The pharmaceutical formulations comprising one or more disclosed nucleic acid constructs, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product. The active agents described herein also can be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption. Such methods for creating liquid, solid, semi-solid, gel, powder or inhalable formulations and the like are known in the art. Techniques for formulation and administration can be found in “Remington: The Science and Practice of Pharmacy” (20th edition, Gennaro (ed.) and Gennaro, Lippincott, Williams & Wilkins, 2000). Alternatively, the inventive compounds can be fused to microspheres in suspension for intravenous injection.

Dosages and regimens for administration are determined by the person of skill, including physicians. Administration of compositions, including the nucleic acid, peptide, composition and cells of the invention can be performed a single time, or repeated at intervals, such as by continuous infusion over a period of time, four times daily, twice daily, daily, every other day, weekly, monthly, or any interval to be determined by the skilled artisan based on the subject involved. Treatment can involve administration over a period of one day only, a week, a month, several months, years, or over a lifetime. Regimens and duration can vary according to any system known in the art, as is known to the skilled person.

Cells expressing a DNA or an mRNA, or naked DNA or RNA in a nanocarrier-type pharmaceutical vehicle, can be injected into a patient, intravenously or into the tissues and/or organs affected by the disease condition to be treated. Current cell vehicles available for human therapy include tolerogenic or immunogenic dendritic cells, and stromal cells. Precursors of certain types of stromal cells (mesenchymal stromal cells or mesenchymal stem cells) may be derived from bone marrow or adipose tissue. The nanocarrier vehicle can be a liposome, a nanoparticle or microparticle, which can be taken up by APCs in vivo.

Doses of the disclosed nucleic acid construct(s), peptide and cells can be determined by the skilled artisan based on the condition of the subject and the route of administration to be used, but are expected to range from about 100 ug to about 10 mg, preferably from about 500 ug to about 10 mg, or about 1 mg to about 10 mg, or about 1 mg to about 5 mg or about 5 mg to about 10 mg and most preferably from about 1 mg to about 5 mg. Optimization/pharmacokinetics can make lower doses effective, therefore even lower doses are contemplated for use with the invention, for example about 10 ug to about 100 ug.

### EXAMPLES

### Example 1. To Assess T Cell Responses to Endotope-Encoded Epitopes In Vitro

Presentation of epitopes can be tested in vitro using T cell clones from T cell receptor transgenic mice such as BDC2.5, BDC12-4.1, NY8.3, G9C8 and G286. Spleen and pooled lymph nodes from these mice are produced into single cell suspensions and Ag-specific CD4+CD25− or CD8+ T cells are purified and co-cultured in vitro with stromal cells modified by lentiviral transduction, plasmid DNA transfection or mRNA-NP transfection to express nucleic acid constructs. T cell responses are measured 3 days later to measure stimulation, markers of anergy and induction of regulatory T cells expressing Foxp3 or IL-10 for example. Prior to adding T cells, stromal cells can be modified to express epitopes, STAT1c and/or conditioned with IFNγ for 3-4 days.

Mice: All mouse strains are purchased from the Jackson Laboratory and bred in our barrier facility: NOD (#001976), NOD.SCID (#001303), NOD.Thy 1.1 (#004483), NOD.CD45.2 (#14149) and T-cell receptor transgenic (TCR-Tg) mice: BDC2.5 (#004460), BDC12-4.1 (#006303/006304) and NY8.3 (#005868). TCR-Tg T cells from these mice respectively recognize the p79/2.5 mimotope (2.5 mi) (23), InsB₉₋₂₃ epitopes and mimotopes (24), and IGRP₂₀₆₋₂₁₄ epitope, all encoded by our NOD mouse-tailored Endotope constructs.

Transduction and mRNA transfection: If a viral vector is used to transduce stromal cells, preferably a multiplicity of infection (MOI; estimated number of. viral particles in suspension) of about 5-10 MOI is used (these cells transduce with high efficiency). Transfection with mRNA-NPs can achieve very high efficiency levels as well (>90%) (21).

### Example 2: To Assess Targeted Non-Viral Gene Delivery to Stromal Cells

In vitro transcribed (IVT) GFP mRNA+miR-142T is custom-synthesized by TriLink. The mRNA is complexed as mRNA-NPs using commercially available in vivo-JetRNA (Polyplus)²². Nanoparticles produced with the different mRNAs are assessed by NanoSight or Zetasizer for consistency in size and particle charge. NOD mice (6-10 weeks of age) are injected intraperitoneally with mRNA-NPs (20 ug mRNA per mouse). Spleen, various lymph nodes and liver (also containing different types of stromal cells shown to mediate tolerance 8.26.41.42) are collected 8h, 24 h and 48h later, digested to release stromal cells, and depleted of lymphocytes by magnetic separation to enrich LNSC and DC populations (<5% of the cellularity in lymphoid tissues). Various APCs are analyzed by flow cytometry to assess expression of GFP alongside LNSC and DC markers (CD45, CD31, Pdpn, CD11c, CD11b, B220, CD317, CD8a). Analysis is repeated using the intravenous and subcutaneous routes of delivery. If high expression is still seen after 48h, later time points are included in the analysis to determine the duration of expression. It is expected that expression will be restricted to stromal cells when using miR-142T.

### Example 3: To Characterize T Cell Responses to Ags Expressed by Stromal Cells

IVT mRNA is produced expressing (1) multiple B-cell epitopes (Endotopc)¹⁹ and (2) mouse STAT1c, both with miR-142T sites (TriLink). Endotope mRNA (5 ug/mouse) combined with GFP or STAT1c mRNA (20 ug/mouse) is formulated as mRNA-NP using in vivo-JetRNA and injected into NOD mice. In the adoptive transfer model, cell proliferation dye-labeled CD4+CD25-T cells and CD8+ T cells (from CD45.2 congenic BDC2.5 and NY8.3 mice, respectively) are injected into NOD mice, that react to two of the mRNA-encoded epitopes (FIG. 1A) and assess Ag-specific T cell responses by flow cytometry at two time points (3 days and 2 weeks later) in terms of clonal frequency (% CD45.2+among total T cells), proliferation (tracer dye dilution) and phenotypic markers (e.g. CD25, CD44, PD-1, Lag-3, CD49b, CD73, FR4, Tim-3 and Tigit). In addition, intracellular staining for Foxp3, IFNγ and IL-10 is performed. In the second model, polyclonal Ag-specific T cell responses to two epitopes is assessed using MHC tetramers (from the NIH Tetramer Core Facility) as previously done20.21.43, using the same time points and analysis panels. In both models, analysis of host T cells (CD45.1+) or tetramer-negative T cells as internal controls provides the baseline expression of those markers on the general T cell population. Three routes of mRNA-NP injection (i.p., i.v., s.c.) are tested, and at least 5 mice per construct (Ag/GFP vs Ag/STAT1c), per time point and per route. STAT1c co-delivery enhances T cell engagement by stromal cells and their expression of tolerance-associated markers.

### Example 4. Validation of Cell Selective Expression with miR-142T

FRCs were transfected with 0.1 ug mRNA/well and analyzed after 48h. THP-1 cells and BM-DCs were transfected with 0.2 ug mRNA/well and analyzed after 48h (THP-1) or 24h (BM-DCs). Mice were injected intraperitoneally with 20-22.4 ug mRNA/mouse, and pancreatic lymph nodes and spleen were processed and digested for analysis after 48h.

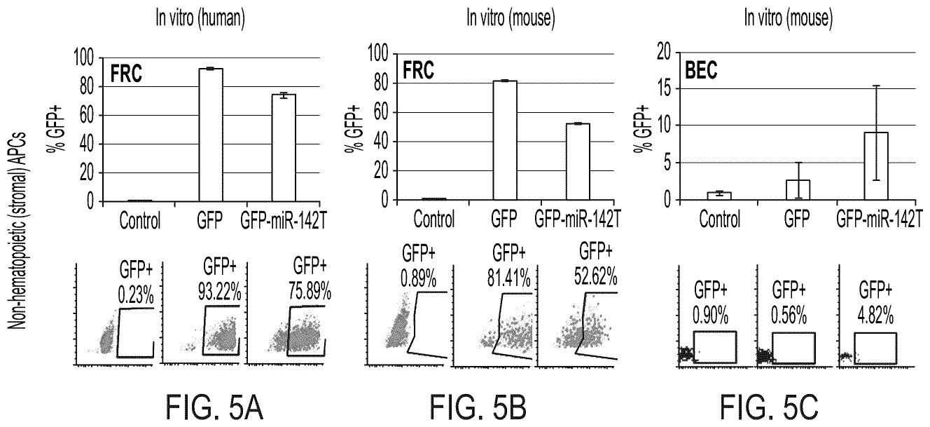

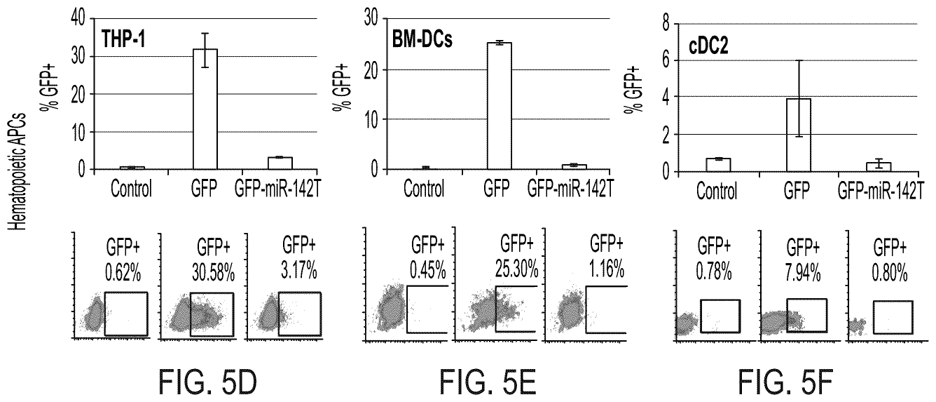

Lymph node stromal APCs include human fibroblastic reticular cells (FRCs) in vitro (FIG. 5A), mouse FRCs in vitro (FIG. 5B) and mouse blood endothelial cells (BEC) in vivo (FIG. 5C). Hematopoietic APCs included human monocytic THP-1 cells (FIG. 5D), mouse bone marrow-derived dendritic cells (DM-DCs) (FIG. 5E) and mouse CD11c+CD11b+cDC2 cells in vivo (FIG. 5F). FIGS. 5A-5B show that the GFP-miR-142T construct is taken into and expressed in stromal APCs in vitro. FIG. 5C shows that the GFP-miR-142 construct is preferentially expressed by stromal APCs in vivo. FIGS. 5D and 5E show that the GFP-miR-142 construct is not expressed in hematopoietic APCs in vitro. FIG. 5F shows that the GFP-miR-142 construct is not expressed in hematopoietic APCs in vivo. Overall, data in FIG. 5 confirm that while GFP mRNA can be expressed in both hematopoietic and stromal APCs, GFP-miR-142T mRNA is selectively expressed in stromal APCs.

### Example 5: To Determine the Efficacy of the mRNA Vaccine in Preventing TID

In the colony used, female NOD mice develop diabetes around 12 weeks of age with ˜90% incidence by 25 weeks of age. Four groups of mice are used: saline (A), mRNA-NPs with GFP mRNA (B), Ag/GFP mRNA (C) and Ag/STAT1c mRNA (D), all with mRNA containing miR142T. Mice are treated every other week (4 injections starting at 8 wks of age) by at least one of the previously tested routes of injection and their glycemia is monitored up to 30 weeks of age to determine incidence of disease as previously reported^{20,43}. Groups of 12 mice are sufficient for an effect size of 20% at 80% power and 0.05 significance. mRNA-NP with Ag/STAT1c mRNA provides the best protection from diabetes development. As alternative to Endotope, proinsulin mRNA is considered as Ag (often used for ASIT in NOD mice). If possible, in addition to preventive treatment from 8 weeks of age, mice are treated later as they reach dysglycemia (150-250 mg/dL) prior to onset of hyperglycemia (>250 mg/dL).

### REFERENCES

1. Katsarou, A., Gudbjornsdottir, S., Rawshani, A., Dabelca, D., Bonifacio, E., Anderson, B. J., Jacobsen, L. M., Schatz, D. A. & Lernmark, A. Type 1 diabetes mellitus. Nat Rev Dis Primers 3, 17016 (2017).
2. Imperatore, G., Boyle, J. P., Thompson, T. J., Case, D., Dabelea, D., Hamman, R. F., Lawrence, J. M., Liese, A. D., Liu, L. L., Mayer-Davis, E. J., Rodriguez, B. L., Standiford, D. & Group, S.f.D.i.Y.S. Projections of type 1 and type 2 diabetes burden in the U.S. population aged <20 years through 2050: dynamic modeling of incidence, mortality, and population growth. Diabetes Care 35, 2515-2520 (2012).
3. Patterson, C. C., Dahlquist, G. G., Gyurus, E., Green, A., Soltesz, G. & Group, E. S. Incidence trends for childhood type 1 diabetes in Europe during 1989-2003 and predicted new cases 2005-20: a multicentre prospective registration study. Lancet 373, 2027-2033 (2009).
4. Coppieters, K. T., Harrison, L. C. & von Herrath, M. G. Trials in type 1 diabetes: Antigen-specific therapies. Clin Immunol 149, 345-355 (2013).
5. Roep, B. O., Wheeler, D. C. S. & Peakman, M. Antigen-based immune modulation therapy for type 1 diabetes: the era of precision medicine. Lancet Diabetes Endocrinol 7, 65-74 (2019).
6. Atkinson, M.A., Roep, B. O., Posgai, A., Wheeler, D. C. S. & Peakman, M. The challenge of modulating beta-cell autoimmunity in type 1 diabetes. Lancet Diabetes Endocrinol 7, 52-64 (2019).
7. Creusot, R. J., Postigo-Fernandez, J. & Teteloshvili, N. Altered Function of Antigen-Presenting Cells in Type 1 Diabetes: A Challenge for Antigen-Specific Immunotherapy? Diabetes 67, 1481-1494 (2018).
8. Kambayashi, T. & Laufer, T. M. Atypical MHC class II-expressing antigen-presenting cells: can anything replace a dendritic cell? Nat Rev Immunol 14, 719-730 (2014).
9. Fletcher, A. L., Lukacs-Kornek, V., Reynoso, E. D., Pinner, S. E., Bellemare-Pelletier, A., Curry, M. S., Collier, A. R., Boyd, R. L. & Turley, S. J. Lymph node fibroblastic reticular cells directly present peripheral tissue antigen under steady-state and inflammatory conditions. J Exp Med 207, 689-697 (2010).
10. Hirosue, S. & Dubrot, J. Modes of Antigen Presentation by Lymph Node Stromal Cells and Their Immunological Implications. Front Immunol 6, 446 (2015).
11. Lee, J., Epardaud, M., Sun, J., Becker, J., Cheng, A., Yonekura, A., Heath, J. & Turley, S. Peripheral antigen display by lymph node stroma promotes T cell tolerance to intestinal self. Nat Immunol 8, 181-190 (2007).
12. Nichols, L., Chen, Y., Colella, T., Bennett, C., Clausen, B. & Engelhard, V. Deletional self-tolerance to a melanocyte/melanoma antigen derived from tyrosinase is mediated by a radio-resistant cell in peripheral and mesenteric lymph nodes. J Immunol 179, 993-1003 (2007).
13. Cohen, J. N., Guidi, C. J., Tewalt, E. F., Qiao, H., Rouhani, S. J., Ruddell, A., Farr, A. G., Tung, K. S. & Engelhard, V. H. Lymph node-resident lymphatic endothelial cells mediate peripheral tolerance via Aire-independent direct antigen presentation. J Exp Med 207, 681-688 (2010).
14. Tewalt, E. F., Cohen, J. N., Rouhani, S. J., Guidi, C. J., Qiao, H., Fahl, S. P., Conaway, M. R., Bender, T. P., Tung, K. S., Vella, A. T., Adler, A. J., Chen, L. & Engelhard, V. H. Lymphatic endothelial cells induce tolerance via PD-L1 and lack of costimulation leading to high-level PD-1 expression on CD8 T cells. Blood 120, 4772-4782 (2012).
15. Cire, S., Da Rocha, S., Ferrand, M., Collins, M. K. & Galy, A. In Vivo Gene Delivery to Lymph Node Stromal Cells Leads to Transgene-specific CD8+ T Cell Anergy in Mice. Mol Ther (2016).
16. Baptista, A. P., Roozendaal, R., Reijmers, R. M., Koning, J. J., Unger, W. W., Greuter, M., Keuning, E. D., Molenaar, R., Goverse, G., Sneeboer, M. M., den Haan, J. M., Boes, M. &

Mebius, R. E. Lymph node stromal cells constrain immunity via MHC class II self-antigen presentation. Elife 3(2014).
17. Dubrot, J., Duraes, F. V., Harle, G., Schlaeppi, A., Brighouse, D., Madelon, N., Gopfert, C., Stokar-Regenscheit, N., Acha-Orbea, H., Reith, W., Gannage, M. & Hugues, S. Absence of MHC-II expression by lymph node stromal cells results in autoimmunity. Life Sci Alliance 1, e201800164 (2018).
18. Postigo-Fernandez, J., Farber, D. L. & Creusot, R. J. Phenotypic alterations in pancreatic lymph node stromal cells from human donors with type 1 diabetes and NOD mice. Diabetologia 62, 2040-2051 (2019).
19. Dastagir, S. R., Postigo-Fernandez, J., Xu, C., Stoeckle, J. H., Firdessa-Fite, R. & Creusot, R. J. Efficient Presentation of Multiple Endogenous Epitopes to Both CD4+ and CD8+Diabetogenic T Cells for Tolerance. Mol Ther Methods Clin Dev 4, 27-38 (2017).
20. Postigo-Fernandez, J. & Creusot, R. J. A multi-epitope DNA vaccine enables a broad engagement of diabetogenic T cells for tolerance in Type 1 diabetes. J Autoimmun 98, 13-23 (2019).
21. Firdessa-Fite, R. & Creusot, R. J. Nanoparticles versus Dendritic Cells as Vehicles to Deliver mRNA Encoding Multiple Epitopes for Immunotherapy. Mol Ther Methods Clin Dev 16, 50-62 (2020).
22. Firdessa-Fite, R., Postigo-Fernandez, J., Toussaint-Moreau, V., Stock, F., Nyamay′ Antu, A., Erbacher, P. & Creusot, R. J. Promising non-viral vector for efficient and versatile delivery of mRNA for antigen-specific immunotherapy. Cell Gene Ther. Insights 6, 1399-1409 (2020).
23. Gonzalez Badillo, F., Zisi Tegou, F., R., M., S., W., Scully, M., Harwell, L., Lupp, M., Postigo-Fernandez, J., Creusot, R. J. & Tomei, A. A. Tissue-Engineered Stromal Reticula to Study Lymph Node Fibroblastic Reticular Cells in Type I Diabetes. Cellular and Molecular Bioengineering (2020).
24. Kariko, K., Muramatsu, H., Welsh, F. A., Ludwig, J., Kato, H., Akira, S. & Weissman, D. Incorporation of pseudouridine into mRNA yields superior nonimmunogenic vector with increased translational capacity and biological stability. Mol Ther 16, 1833-1840 (2008).
25. Brown, B.D., Venneri, M.A., Zingale, A., Sergi Sergi, L. & Naldini, L. Endogenous microRNA regulation suppresses transgene expression in hematopoietic lineages and enables stable gene transfer. Nat Med 12, 585-591 (2006).
26. Akbarpour, M., Goudy, K. S., Cantore, A., Russo, F., Sanvito, F., Naldini, L., Annoni, A. & Roncarolo, M. G. Insulin B chain 9-23 gene transfer to hepatocytes protects from type 1 diabetes by inducing Ag-specific FoxP3+ Tregs. Sci Transl Med 7, 289ra281 (2015).
27. Annoni, A., Brown, B.D., Cantore, A., Sergi, L. S., Naldini, L. & Roncarolo, M. G. In vivo delivery of a microRNA-regulated transgene induces antigen-specific regulatory T cells and promotes immunologic tolerance. Blood 114, 5152-5161 (2009).
28. Stagg, J., Pommey, S., Eliopoulos, N. & Galipeau, J. Interferon-gamma-stimulated marrow stromal cells: a new type of nonhematopoietic antigen-presenting cell. Blood 107, 2570-2577 (2006).
29. Duijvestein, M., Wildenberg, M. E., Welling, M. M., Hennink, S., Molendijk, I., van Zuylen, V. L., Bosse, T., Vos, A. C., de Jonge-Muller, E. S., Roelofs, H., van der Weerd, L., Verspaget, H. W., Fibbe, W. E., te Velde, A. A., van den Brink, G. R. & Hommes, D. W. Pretreatment with interferon-gamma enhances the therapeutic activity of mesenchymal stromal cells in animal models of colitis. Stem Cells 29, 1549-1558 (2011).
30. van Megen, K. M., van 't Wout, E. T., Lages Motta, J., Dekker, B., Nikolic, T. & Roep, B. O. Activated Mesenchymal Stromal Cells Process and Present Antigens Regulating Adaptive Immunity. Front Immunol 10, 694 (2019).
31. Sheng, H., Wang, Y., Jin, Y., Zhang, Q., Zhang, Y., Wang, L., Shen, B., Yin, S., Liu, W., Cui, L. & Li, N. A critical role of IFNgamma in priming MSC-mediated suppression of T cell proliferation through up-regulation of B7-H1. Cell Res 18, 846-857 (2008).
32. Lane, R. S., Femel, J., Breazeale, A. P., Loo, C. P., Thibault, G., Kaempf, A., Mori, M., Tsujikawa, T., Chang, Y. H. & Lund, A. W. IFNgamma-activated dermal lymphatic vessels inhibit cytotoxic T cells in melanoma and inflamed skin. J Exp Med 215, 3057-3074 (2018).
33. Kim, D. S., Jang, I. K., Lee, M. W., Ko, Y. J., Lee, D. H., Lee, J. W., Sung, K. W., Koo, H. H. & Yoo, K. H. Enhanced Immunosuppressive Properties of Human Mesenchymal Stem Cells

Primed by Interferon-gamma. EBioMedicine 28, 261-273 (2018).
34. Meisel, R., Zibert, A., Laryea, M., Gobel, U., Daubener, W. & Dilloo, D. Human bone marrow stromal cells inhibit allogeneic T-cell responses by indoleamine 2,3-dioxygenase-mediated tryptophan degradation. Blood 103, 4619-4621 (2004).
35. Lukacs-Kornek, V., Malhotra, D., Fletcher, A. L., Acton, S. E., Elpek, K. G., Tayalia, P., Collier, A. R. & Turley, S. J. Regulated release of nitric oxide by nonhematopoietic stroma controls expansion of the activated T cell pool in lymph nodes. Nat Immunol 12, 1096-1104 (2011).
36. Siegert, S., Huang, H. Y., Yang, C. Y., Scarpellino, L., Carrie, L., Essex, S., Nelson, P. J., Heikenwalder, M., Acha-Orbea, H., Buckley, C. D., Marsland, B. J., Zehn, D. & Luther, S. A. Fibroblastic reticular cells from lymph nodes attenuate T cell expansion by producing nitric oxide. PLOS One 6, e27618 (2011).
37. Scotto, M., Afonso, G., Larger, E., Raverdy, C., Lemonnier, F. A., Carel, J. C., Dubois-Laforgue, D., Baz, B., Levy, D., Gautier, J. F., Launay, O., Bruno, G., Boitard, C., Sechi, L. A., Hutton, J. C., Davidson, H. W. & Mallone, R. Zinc transporter (ZnT)8(186-194) is an immunodominant CD8+ T cell epitope in HLA-A2+type 1 diabetic patients. Diabetologia 55, 2026-2031 (2012).
38. Seay, H. R., Yusko, E., Rothweiler, S. J., Zhang, L., Posgai, A. L., Campbell-Thompson, M., Vignali, M., Emerson, R. O., Kaddis, J. S., Ko, D., Nakayama, M., Smith, M. J., Cambier, J. C., Pugliese, A., Atkinson, M.A., Robins, H. S. & Brusko, T. M. Tissue distribution and clonal diversity of the T and B cell repertoire in type 1 diabetes. JCI Insight 1, e88242 (2016).
39. Yang, J., Wen, X., Xu, H., Torres-Chinn, N., Speake, C., Greenbaum, C. J., Nepom, G. T. & Kwok, W. W. Antigen-Specific T Cell Analysis Reveals That Active Immune Responses to beta Cell Antigens Are Focused on a Unique Set of Epitopes. J Immunol (2017).
40. James, E. A., Mallone, R., Kent, S. C. & DiLorenzo, T. P. T-Cell Epitopes and Neo-epitopes in Type 1 Diabetes: A Comprehensive Update and Reappraisal. Diabetes 69, 1311-1335 (2020).
41. Carambia, A., Freund, B., Schwinge, D., Heine, M., Laschtowitz, A., Huber, S., Wraith, D. C., Korn, T., Schramm, C., Lohse, A. W., Heeren, J. & Herkel, J. TGF-beta-dependent induction of CD4(+)CD25(+)Foxp3(+) Tregs by liver sinusoidal endothelial cells. J Hepatol 61, 594-599 (2014).
42. Xu, X., Jin, R., Li, M., Wang, K., Zhang, S., Hao, J., Sun, X., Zhang, Y., Wu, H., Zhang, J. & Ge, Q. Liver sinusoidal endothelial cells induce tolerance of autoreactive CD4+recent thymic emigrants. Sci Rep 6, 19861 (2016).
43. Firdessa-Fite, R., Johnson, S. N., Leon, M.A., Khosravi-Maharlooei, M., Baker, R. L., Sestak, J. O., Berkland, C. & Creusot, R. J. Soluble Antigen Arrays Efficiently Deliver Peptides and Arrest Spontaneous Autoimmune Diabetes. Diabetes (2021).
44. Non-Genetically Encoded Epitopes Are Relevant Targets in Autoimmune Diabetes.Nguyen H, Guyer P, Ettinger R A, James E A. Biomedicines. 2021 Feb. 17;9(2):202. doi: 10.3390/biomedicines9020202.PMID: 33671312
45. T-cell responses to hybrid insulin peptides prior to type 1 diabetes development. Mitchell A M, Alkanani A A, McDaniel K A, Pyle L, Waugh K, Steck A K, Nakayama M, Yu L, Gottlieb P A, Rewers M J, Michels A W.Proc Natl Acad Sci USA. 2021 Feb. 9;118(6):e2019129118. doi: 10.1073/pnas.2019129118.PMID: 33542101
46. Neoepitopes in Type 1 Diabetes: Etiological Insights, Biomarkers and TherapeuticTargets.Rodriguez-Calvo T, Johnson J D, Overbergh L, Dunne J L.Front Immunol. 2021 Apr. 19; 12:667989. doi: 10.3389/fimmu.2021.667989. eCollection 2021.PMID: 33953728

This invention is not limited to the particular processes, compositions, or methodologies described, as these may vary. The terminology used in the description is for the purpose of describing the particular versions or embodiments only, and is not intended to limit the scope of the present invention which will be limited only by the appended claims. Although any methods and materials similar or equivalent to those described herein can be used in the practice or testing of embodiments of the present invention, the preferred methods, devices, and materials are now described. All publications mentioned herein, are incorporated by reference in their entirety; nothing herein is to be construed as an admission that the invention is not entitled to antedate such disclosure by virtue of prior invention.

1. Two or more nucleic acid constructs for administration into a subject comprising (a) a first nucleic acid construct that comprises at least one nucleic acid sequence encoding at least one endosome-targeting signal, at least one nucleic acid sequence encoding at least one CD4 epitope, at least one nucleic acid sequence encoding at least one cleavage site, at least one nucleic acid sequence encoding at least one CD8 epitope, and at least one miR142 target site nucleic acid sequence; and (b) a second nucleic acid construct that comprises at least one nucleic acid sequence encoding STAT1 and at least one miR142 target site nucleic acid sequence.

2. The nucleic acid constructs of claim 1, which are DNA.

3. The nucleic acid constructs of claim 1, which are mRNA.

4. The nucleic acid constructions of claim 1, wherein the at least one CD4 epitope or CD8 epitope comprises at least a portion of an antigen involved in an autoimmune disease condition.

5. The nucleic acid constructs of claim 1, wherein the antigen is at least one selected from Proinsulin, insulin, GAD65, IA-2, IGRP, ZnT8, hybrid insulin peptide, Charmogranin A, IAPP, ICA69, IA-2B, RegII, GPR78, HSP60, HSP70, SEQ ID NOs: 11-17, myelin basic protein, proteolipid protein, myelin oligodendrocyte glycoprotein, minor antigens, myelin-associated antigen, myelin-associated oligodendrocyte basic protein, 2′, 3′-cyclic-nucleotide 3′-phosphodiesterase, S100β, transaldolase, collagen, cartilage glycoprotein 39, aggrecan G1, rheumatoid factor, citrullinated peptides, carbamylated antigens, PAD4, BRAF, HSP65, basement membrane laminin, LL37, progranulin, desmoglein 3, Pso p27, Ezrin, maspin, peroxiredoxin 2, HSP 27, Collagen type XVII, Keratin 13, hnRNP-A1, FLJ00294, hnRNP-C1/C2, SCG, GLCDAC05, alpha-endosulfine, NOL8, GFGR3, dematin, signal recognition particle subunit 14, EPF, CUZDI, GP2, HMGB1/HMGB2, ASCA autoantibodies, FAM84A, Collagen type VII, Complement C3, Ubiquitination factor e4A (UBE4A), CBirl flagellin autoantibodies, Alpha 3(IV)NC1, BP180 autoantibodies, Galectin-3 autoantibodies, Catalase, alpha-enolase, or Lactoferrin autoantibodies.

6. The nucleic acid constructs of claim 1, wherein the autoimmune disease condition is selected from a group consisting of type 1 diabetes, rheumatoid arthritis, multiple sclerosis, psoriasis, Crohn's disease and ulcerative colitis.

7. The nucleic acid constructs of claim 1, wherein the amino acid sequence of at least one CD4 epitope is selected from SEQ ID NOs:31, 33, 35, 37, 39, 41 or 43, or a sequence comprising at least 95% identity therewith.

8. The nucleic acid constructs of claim 1, wherein the amino acid sequence of the at least one CD8 epitope is selected from SEQ ID NOs: 53, 55, 56, or 57, or a sequence comprising at least 95% identity therewith.

9. The nucleic acid constructs of claim 1, wherein the sequence of STAT1 is mutated to prevent dephosphorylation.

10. The nucleic acid constructs claim 1, wherein the sequence of STAT1 is selected from SEQ ID NOs: 1 or 2, or a sequence comprising at least 95% identity therewith.

11. The nucleic acid constructs of claim 1, wherein the miR142 target site nucleic acid sequence is selected from SEQ ID NOs:9 or 10, or a sequence comprising at least 95% identity therewith.

12. An isolated nucleic acid construct that comprises at least one nucleic acid sequence encoding at least one endosome-targeting signal, at least one nucleic acid sequence encoding at least one CD4 epitope, at least one nucleic acid sequence encoding at least one cleavage site, at least one nucleic acid sequence encoding at least one CD8 epitope; at least one nucleic acid sequence encoding STAT1; and at least one miR142 target site nucleic acid sequence.

13. The nucleic acid construct of claim 12, which is DNA.

14. The nucleic acid construct of claim 12, which is mRNA.

15. The nucleic acid construct of claim 12, wherein the sequences are in the order from 5′ to 3′: at least one nucleic acid sequence encoding at least one endosome-targeting signal, at least one nucleic acid sequence encoding at least one CD4 epitope, at least one nucleic acid sequence encoding at least one cleavage site, at least one nucleic acid sequence encoding at least one CD8 epitope; at least one nucleic acid sequence encoding STAT1; and at least one miR142 target site nucleic acid sequence.

16. The nucleic acid construct of claim 12, the at least one CD4 epitope or CD8 epitope comprises at least a portion of an antigen involved in an autoimmune disease condition.

17. The nucleic acid construct of claim 12, wherein the antigen is at least one selected from Table 1.

18. The nucleic acid construct of claim 12, wherein the autoimmune disease condition is selected from the group consisting of type 1 diabetes, rheumatoid arthritis, multiple sclerosis, psoriasis, Crohn's disease and ulcerative colitis.

19. The nucleic acid constructs of claim 12, wherein the sequence of CD4 epitope is selected from SEQ ID NOs: 31, 33, 35, 37, 39, 41 or 43, or a sequence comprising at least 95% identity therewith.

20. The nucleic acid constructs of claim 12, wherein the sequence of CD8 epitope is selected from SEQ ID NOs: 53, 55, 56, or 57, or a sequence comprising at least 95% identity therewith.

21. The nucleic acid construct of claim 12, wherein the sequence of STAT1 is selected from SEQ ID NOs: 1 or 2, or a sequence comprising at least 95% identity therewith.

22. The nucleic acid construct of claim 12, wherein the sequence of a miR142 target site is selected from SEQ ID NOs: 9 or 10, or a sequence comprising at least 95% identity therewith.

23. A nanoparticle comprising the nucleic acid constructs of claim 1 and optionally at least one polycationic molecule complexed therewith or liposome.

24. The nanoparticle of claim 23, wherein the polycationic molecule is a positively charged cell-penetrating peptide (CPP).

25. The nanoparticle of claim 24, wherein the CPP is polylysine, polyarginine or Tat peptide.

26. The nanoparticle of claim 23, wherein the polycationic molecule is a polycationic polymer.

27. The nanoparticle of claim 26, wherein the polycationic polymer is polyethylenimine.

28. The nanoparticle of claim 23, wherein the polycationic molecule is a polycationic lipid.

29. An immunogenic composition comprising the nanoparticle of claim 23 and an optionally a pharmaceutically acceptable carrier.

30. A method for inducing immune tolerance in a subject with an autoimmune disease condition comprised of administering an effective amount of the nucleic acid constructs of claim 1, to the subject.

31. The method of claim 30, wherein the effective amount is administered orally or intramuscularly, subcutaneously/intradermally or intravenously.

32. The method of claim 30, wherein the autoimmune disease condition is selected from the group consisting of type 1 diabetes, rheumatoid arthritis, multiple sclerosis, psoriasis, Crohn's disease and ulcerative colitis.


SEQUENCE LISTING (62 sequences)

SEQ ID NO: 1 - DNA, 2253 bp, Homo sapiens
atgtctcagtggtacgaacttcagcagcttgactcaaaattcctggagcaggttcaccagctttatgatgacagttttcccatggaaatcagacagtacctggcacagtggttagaaaagcaagactgggagcacgctgccaatgatgtttcatttgccaccatccgttttcatgacctcctgtcacagctggatgatcaatatagtcgcttttctttggagaataacttcttgctacagcataacataaggaaaagcaagcgtaatcttcaggataattttcaggaagacccaatccagatgtctatgatcatttacagctgtctgaaggaagaaaggaaaattctggaaaacgcccagagatttaatcaggctcagtcggggaatattcagagcacagtgatgttagacaaacagaaagagcttgacagtaaagtcagaaatgtgaaggacaaggttatgtgtatagagcatgaaatcaagagcctggaagatttacaagatgaatatgacttcaaatgcaaaaccttgcagaacagagaacacgagaccaatggtgtggcaaagagtgatcagaaacaagaacagctgttactcaagaagatgtatttaatgcttgacaataagagaaaggaagtagttcacaaaataatagagttgctgaatgtcactgaacttacccagaatgccctgattaatgatgaactagtggagtggaagcggagacagcagagcgcctgtattggggggccgcccaatgcttgcttggatcagctgcagaactggttcactatagttgcggagagtctgcagcaagttcggcagcagcttaaaaagttggaggaattggaacagaaatacacctacgaacatgaccctatcacaaaaaacaaacaagtgttatgggaccgcaccttcagtcttttccagcagctcattcagagctcgtttgtggtggaaagacagccctgcatgccaacgcaccctcagaggccgctggtcttgaagacaggggtccagttcactgtgaagttgagactgttggtgaaattgcaagagctgaattataatttgaaagtcaaagtcttatttgataaagatgtgaatgagagaaatacagtaaaaggatttaggaagttcaacattttgggcacgcacacaaaagtgatgaacatggaggagtccaccaatggcagtctggcggctgaatttcggcacctgcaattgaaagaacagaaaaatgctggcaccagaacgaatgagggtcctctcatcgttactgaagagcttcactcccttagttttgaaacccaattgtgccagcctggtttggtaattgacctcgagacgacctctctgcccgttgtggtgatctccaacgtcagccagctcccgagcggttgggcctccatcctttggtacaacatgctggtggcggaacccaggaatctgtccttcttcctgactccaccatgtgcacgatgggctcagctttcagaagtgctgagttggcagttttcttctgtcaccaaaagaggtctcaatgtggaccagctgaacatgttgggagagaagcttcttggtcctaacgccagccccgatggtctcattccgtggacgaggttttgtaaggaaaatataaatgataaaaattttcccttctggctttggattgaaagcatcctagaactcattaaaaaacacctgctccctctctggaatgatgggtgcatcatgggcttcatcagcaaggagcgagagcgtgccctgttgaaggaccagcagccggggaccttcctgctgcggttcagtgagagctcccgggaaggggccatcacattcacatgggtggagcggtcccagaacggaggcgaacctgacttccatgcggttgaaccctacacgaagaaagaactttctgctgttactttccctgacatcattcgcaattacaaagtcatggcttgtgagtgtattcctgagaatcccctgaagtatctgtatccaaatattgacaaagaccatgcctttggaaagtattactccaggccaaaggaagcaccagagccaatggaacttgatggccctaaaggaactggatatatcaagactgagttgatttctgtgtctgaagttcacccttctagacttcagaccacagacaacctgctccccatgtctcctgaggagtttgacgaggtgtctcggatagtgggctctgtagaattcgacagtatgatgaacacagtatag

SEQ ID NO: 2 - DNA, 2268 bp, Mus musculus
atgtcacagtggttcgagcttcagcagctggactccaagttcctggagcaggtccaccagctgtacgatgacagtttccccatggaaatcagacagtacctggcccagtggctggaaaagcaagactgggagcacgctgcctatgatgtctcgtttgcgaccatccgcttccatgacctcctctcacagctggacgaccagtacagccgcttttctctggagaataatttcttgttgcagcacaacatacggaaaagcaagcgtaatctccaggataacttccaagaagatcccgtacagatgtccatgatcatctacaactgtctgaaggaagaaaggaagattttggaaaatgcccaaagatttaatcaggcccaggagggaaatattcagaacactgtgatgttagataaacagaaggagctggacagtaaagtcagaaatgtgaaggatcaagtcatgtgcatagagcaggaaatcaagaccctagaagaattacaagatgaatatgactttaaatgcaaaacctctcagaacagagaaggtgaagccaatggtgtggcgaagagcgaccaaaaacaggaacagctgctgctccacaagatgtttttaatgcttgacaataagagaaaggagataattcacaaaatcagagagttgctgaattccatcgagctcactcagaacactctgattaatgacgagctcgtggagtggaagcgaaggcagcagagcgcctgcatcgggggaccgcccaacgcctgcctggatcagctgcaaagctggttcaccattgttgcagagaccctgcagcagatccgtcagcagcttaaaaagctggaggagttggaacagaaattcacctatgagcccgaccctattacaaaaaacaagcaggtgttgtcagatcgaaccttcctcctcttccagcagctcattcagagctccttcgtggtagaacgacagccgtgcatgcccactcacccgcagaggcccctggtcttgaagactggggtacagttcactgtcaagctgagactgttggtgaaattgcaagagctgaactataacttgaaagtgaaagtctcatttgacaaagatgtgaacgagaaaaacacagttaaaggatttcggaagttcaacatcttgggtacgcacacaaaagtgatgaacatggaagaatccaccaacggaagtctggcagctgagttccgacacctgcaactgaaggaacagaaaaacgctgggaacagaactaatgaggggcctctcattgtcaccgaagaacttcactctcttagctttgaaacccagttgtgccagccaggcttggtgattgacctggaggtctttgttccctttcagaccacctctcttcctgtcgtggtgatctccaacgtcagccagctccccagtggctgggcgtctatcctgtggtacaacatgctggtgacagagcccaggaatctctccttcttcctgaaccccccgtgcgcgtggtggtcccagctctcagaggtgttgagttggcagttttcatcagtcaccaagagaggtctgaacgcagaccagctgagcatgctgggagagaagctgctgggccctaatgctggccctgatggtcttattccatggacaaggttttgtaaggaaaatattaatgataaaaatttctccttctggccttggattgacaccatcctagagctcattaagaagcacctgctgtgcctctggaatgatgggtgcattatgggcttcatcagcaaggagcgagaacgcgctctgctcaaggaccagcagccagggacgttcctgcttagattcagtgagagctcccgggaaggggccatcacattcacatgggtggaacggtcccagaacggaggtgaacctgacttccatgccgtggagccctacacgaaaaaagaactttcagctgttactttcccagatattattcgcaactacaaagtcatggcttgcgagtgcataccagagaatcccctgaagtatctgtaccccaatattgacaaagaccacgcctttgggaagtattattccagaccaaaggaagcaccagaaccgatggagcttgacgaccctaagcgaactggatacatcaagactgagttgatttctgtgtctgaagtccacccttctagacttcagaccacagacaacctgcttcccatgtctccagaggagtttgatgagatgtcccggatagtgggccccgaatttgacagtatgatgagcacagtataa

SEQ ID NO: 3 - protein, 750 aa, Homo sapiens
MSQWYELQQLDSKFLEQVHQLYDDSFPMEIRQYLAQWLEKQDWEHAANDVSFATIRFHDLLSQLDDQYSRFSLENNFLLQHNIRKSKRNLQDNFQEDPIQMSMIIYSCLKEERKILENAQRFNQAQSGNIQSTVMLDKQKELDSKVRNVKDKVMCIEHEIKSLEDLQDEYDFKCKTLQNREHETNGVAKSDQKQEQLLLKKMYLMLDNKRKEVVHKIIELLNVTELTQNALINDELVEWKRRQQSACIGGPPNACLDQLQNWFTIVAESLQQVRQQLKKLEELEQKYTYEHDPITKNKQVLWDRTFSLFQQLIQSSFVVERQPCMPTHPQRPLVLKTGVQFTVKLRLLVKLQELNYNLKVKVLFDKDVNERNTVKGFRKFNILGTHTKVMNMEESTNGSLAAEFRHLQLKEQKNAGTRTNEGPLIVTEELHSLSFETQLCQPGLVIDLETTSLPVVVISNVSQLPSGWASILWYNMLVAEPRNLSFFLTPPCARWAQLSEVLSWQFSSVTKRGLNVDQLNMLGEKLLGPNASPDGLIPWTRFCKENINDKNFPFWLWIESILELIKKHLLPLWNDGCIMGFISKERERALLKDQQPGTFLLRFSESSREGAITFTWVERSQNGGEPDFHAVEPYTKKELSAVTFPDIIRNYKVMACECIPENPLKYLYPNIDKDHAFGKYYSRPKEAPEPMELDGPKGTGYIKTELISVSEVHPSRLQTTDNLLPMSPEEFDEVSRIVGSVEFDSMMNTV

SEQ ID NO: 4 - protein, 749 aa, Mus musculus
MSQWFELQQLDSKFLEQVHQLYDDSFPMEIRQYLAQWLEKQDWEHAAYDVSFATIRFHDLLSQLDDQYSRFSLENNFLLQHNIRKSKRNLQDNFQEDPVQMSMIIYNCLKEERKILENAQRFNQAQEGNIQNTVMLDKQKELDSKVRNVKDQVMCIEQEIKTLEELQDEYDFKCKTSQNREGEANGVAKSDQKQEQLLLHKMFLMLDNKRKEIIHKIRELLNSIELTQNTLINDELVEWKRRQQSACIGGPPNACLDQLQSWFTIVAETLQQIRQQLKKLEELEQKFTYEPDPITKNKQVLSDRTFLLFQQLIQSSFVVERQPCMPTHPQRPLVLKTGVQFTVKLRLLVKLQELNYNLKVKVSFDKDVNEKNTVKGFRKFNILGTHTKVMNMEESTNGSLAAEFRHLQLKEQKNAGNRTNEGPLIVTEELHSLSFETQLCQPGLVIDLETTSLPVVVISNVSQLPSGWASILWYNMLVTEPRNLSFFLNPPCAWWSQLSEVLSWQFSSVTKRGLNADQLSMLGEKLLGPNAGPDGLIPWTRFCKENINDKNFSFWPWIDTILELIKKHLLCLWNDGCIMGFISKERERALLKDQQPGTFLLRFSESSREGAITFTWVERSQNGGEPDFHAVEPYTKKELSAVTFPDIIRNYKVMACECIPENPLKYLYPNIDKDHAFGKYYSRPKEAPEPMELDDPKRTGYIKTELISVSEVHPSRLQTTDNLLPMSPEEFDEMSRIVGPEFDSMMSTV

SEQ ID NO: 5 - protein, 750 aa, Homo sapiens
MSQWYELQQLDSKFLEQVHQLYDDSFPMEIRQYLAQWLEKQDWEHAANDVSFATIRFHDLLSQLDDQYSRFSLENNFLLQHNIRKSKRNLQDNFQEDPIQMSMIIYSCLKEERKILENAQRFNQAQSGNIQSTVMLDKQKELDSKVRNVKDKVMCIEHEIKSLEDLQDEYDFKCKTLQNREHETNGVAKSDQKQEQLLLKKMYLMLDNKRKEVVHKIIELLNVTELTQNALINDELVEWKRRQQSACIGGPPNACLDQLQNWFTIVAESLQQVRQQLKKLEELEQKYTYEHDPITKNKQVLWDRTFSLFQQLIQSSFVVERQPCMPTHPQRPLVLKTGVQFTVKLRLLVKLQELNYNLKVKVLFDKDVNERNTVKGFRKFNILGTHTKVMNMEESTNGSLAAEFRHLQLKEQKNAGTRTNEGPLIVTEELHSLSFETQLCQPGLVIDLETTSLPVVVISNVSQLPSGWASILWYNMLVAEPRNLSFFLTPPCARWAQLSEVLSWQFSSVTKRGLNVDQLNMLGEKLLGPNASPDGLIPWTRFCKENINDKNFPFWLWIESILELIKKHLLPLWNDGCIMGFISKERERALLKDQQPGTFLLRFSESSREGAITFTWVERSQNGGEPDFHAVEPYTKKELSAVTFPDIIRNYKVMAAENIPENPLKYLYPNIDKDHAFGKYYSRPKEAPEPMELDGPKGTGYIKTELISVSEVHPSRLQTTDNLLPMSPEEFDEVSRIVGSVEFDSMMNTV

SEQ ID NO: 6 - protein, 712 aa, Homo sapiens
MSQWYELQQLDSKFLEQVHQLYDDSFPMEIRQYLAQWLEKQDWEHAANDVSFATIRFHDLLSQLDDQYSRFSLENNFLLQHNIRKSKRNLQDNFQEDPIQMSMIIYSCLKEERKILENAQRFNQAQSGNIQSTVMLDKQKELDSKVRNVKDKVMCIEHEIKSLEDLQDEYDFKCKTLQNREHETNGVAKSDQKQEQLLLKKMYLMLDNKRKEVVHKIIELLNVTELTQNALINDELVEWKRRQQSACIGGPPNACLDQLQNWFTIVAESLQQVRQQLKKLEELEQKYTYEHDPITKNKQVLWDRTFSLFQQLIQSSFVVERQPCMPTHPQRPLVLKTGVQFTVKLRLLVKLQELNYNLKVKVLFDKDVNERNTVKGFRKFNILGTHTKVMNMEESTNGSLAAEFRHLQLKEQKNAGTRTNEGPLIVTEELHSLSFETQLCQPGLVIDLETTSLPVVVISNVSQLPSGWASILWYNMLVAEPRNLSFFLTPPCARWAQLSEVLSWQFSSVTKRGLNVDQLNMLGEKLLGPNASPDGLIPWTRFCKENINDKNFPFWLWIESILELIKKHLLPLWNDGCIMGFISKERERALLKDQQPGTFLLRFSESSREGAITFTWVERSQNGGEPDFHAVEPYTKKELSAVTFPDIIRNYKVMAAENIPENPLKYLYPNIDKDHAFGKYYSRPKEAPEPMELDGPKGTGYIKTELISVSEV

SEQ ID NO: 7 - protein, 755 aa, Mus musculus
MSQWFELQQLDSKFLEQVHQLYDDSFPMEIRQYLAQWLEKQDWEHAAYDVSFATIRFHDLLSQLDDQYSRFSLENNFLLQHNIRKSKRNLQDNFQEDPVQMSMIIYNCLKEERKILENAQRFNQAQEGNIQNTVMLDKQKELDSKVRNVKDQVMCIEQEIKTLEELQDEYDFKCKTSQNREGEANGVAKSDQKQEQLLLHKMFLMLDNKRKEIIHKIRELLNSIELTQNTLINDELVEWKRRQQSACIGGPPNACLDQLQSWFTIVAETLQQIRQQLKKLEELEQKFTYEPDPITKNKQVLSDRTFLLFQQLIQSSFVVERQPCMPTHPQRPLVLKTGVQFTVKLRLLVKLQELNYNLKVKVSFDKDVNEKNTVKGFRKFNILGTHTKVMNMEESTNGSLAAEFRHLQLKEQKNAGNRTNEGPLIVTEELHSLSFETQLCQPGLVIDLEVFVPFQTTSLPVVVISNVSQLPSGWASILWYNMLVTEPRNLSFFLNPPCAWWSQLSEVLSWQFSSVTKRGLNADQLSMLGEKLLGPNAGPDGLIPWTRFCKENINDKNFSFWPWIDTILELIKKHLLCLWNDGCIMGFISKERERALLKDQQPGTFLLRFSESSREGAITFTWVERSQNGGEPDFHAVEPYTKKELSAVTFPDIIRNYKVMAAENIPENPLKYLYPNIDKDHAFGKYYSRPKEAPEPMELDDPKRTGYIKTELISVSEVHPSRLQTTDNLLPMSPEEFDEMSRIVGPEFDSMMSTV

SEQ ID NO: 8 - protein, 749 aa, Mus musculus
MSQWFELQQLDSKFLEQVHQLYDDSFPMEIRQYLAQWLEKQDWEHAAYDVSFATIRFHDLLSQLDDQYSRFSLENNFLLQHNIRKSKRNLQDNFQEDPVQMSMIIYNCLKEERKILENAQRFNQAQEGNIQNTVMLDKQKELDSKVRNVKDQVMCIEQEIKTLEELQDEYDFKCKTSQNREGEANGVAKSDQKQEQLLLHKMFLMLDNKRKEIIHKIRELLNSIELTQNTLINDELVEWKRRQQSACIGGPPNACLDQLQSWFTIVAETLQQIRQQLKKLEELEQKFTYEPDPITKNKQVLSDRTFLLFQQLIQSSFVVERQPCMPTHPQRPLVLKTGVQFTVKLRLLVKLQELNYNLKVKVSFDKDVNEKNTVKGFRKFNILGTHTKVMNMEESTNGSLAAEFRHLQLKEQKNAGNRTNEGPLIVTEELHSLSFETQLCQPGLVIDLETTSLPVVVISNVSQLPSGWASILWYNMLVTEPRNLSFFLNPPCAWWSQLSEVLSWQFSSVTKRGLNADQLSMLGEKLLGPNAGPDGLIPWTRFCKENINDKNFSFWPWIDTILELIKKHLLCLWNDGCIMGFISKERERALLKDQQPGTFLLRFSESSREGAITFTWVERSQNGGEPDFHAVEPYTKKELSAVTFPDIIRNYKVMAAENIPENPLKYLYPNIDKDHAFGKYYSRPKEAPEPMELDDPKRTGYIKTELISVSEVHPSRLQTTDNLLPMSPEEFDEMSRIVGPEFDSMMSTV

SEQ ID NO: 9 - DNA, 23 bp, Artificial Sequence
  miR-142T single site
tccataaagtaggaaacactaca

SEQ ID NO: 10 - DNA, 118 bp, Artificial Sequence
  miR-142T 4X site
ctagagtcgactccataaagtaggaaacactacacgattccataaagtaggaaacactacaaccggttccataaagtaggaaacactacatcactccataaagtaggaaacactacac

SEQ ID NO: 11 - protein, 15 aa, Artificial Sequence
  epitope sequence
SHLVEALYLVCGERG

SEQ ID NO: 12 - protein, 9 aa, Artificial Sequence
  epitope sequence
LYLVCGERG

SEQ ID NO: 13 - protein, 15 aa, Artificial Sequence
  epitope sequence
SHLVEALYLVCGEEG

SEQ ID NO: 14 - protein, 14 aa, Artificial Sequence
  epitope sequence
WSRMDQLAKELTAE

SEQ ID NO: 15 - protein, 10 aa, Artificial Sequence
  epitope sequence
AVPPLWVRME

SEQ ID NO: 16 - protein, 9 aa, Artificial Sequence
  epitope sequence
VYLKTNVFL

SEQ ID NO: 17 - protein, 14 aa, Artificial Sequence
  epitope sequence
KKGAAALGIGTDSV

SEQ ID NO: 18 - protein, 12 aa, Artificial Sequence
  Tat peptide
GRKKRRQRRRPQ

SEQ ID NO: 19-23 - skipped (5 entries)

SEQ ID NO: 24 - DNA, 69 bp, Artificial Sequence
  codon-optimized albumin secretion signal
atgaagtgggtaacctttctcctcctcctcttcatctccggttctgccttttctaggggcaagcttatg

SEQ ID NO: 25 - DNA, 75 bp, Artificial Sequence
  albumin leader sequence
atgaagtgggtaacctttatttcccttctttttctctttagctcggcttattccaggggtgtgtttcgtcgagat

SEQ ID NO: 26 - DNA, 63 bp, Artificial Sequence
  immunoglobulin kappa-chain leader sequence
atggagacagacacactcctgctatgggtactgctgctctgggttccaggttccactggtgac

SEQ ID NO: 27 - DNA, 675 bp, Artificial Sequence
  endotope sequence
actagtgccaccatgatggatcaagctagatcagcattctctaacttgtttggtggagaaccattgtcatatacccggttcagcctggctcggcaagtagatggcgataacagtcatgtggagatgaaacttgctgtagatgaagaagaaaatgctgacaataacacaaaggccaatgtcacaaaaccaaaaaggtgtagtggaagtatctgctatgggactattgctgtgatcgtctttttcttgattggatttatgattggctacttgggctattgtaaaggggtagaaccaaaaactgagtgtgagagactggcaggaaccgagtctccagtgagggaggagccaggagaggacttccctgcaccgcggtgtggttcccacctggtggaggctctctacctggtgtgtggggaggagggcttcttcaagcgagcagttcgacctctatgggtacgtatggaaaagcggtctctcaagaagggagctgcagccttagggattggaacagacagtgtgattctgattgagggcagaggaagtctgctaacatgcggtgacgtcgaggagaatcctggacctgaggctctctacctggtgtgtggggagcgtggcttcttcttgagtgtgtacctgaagaccaacgtcttcctcttcctgcccgggtaggtcgac

SEQ ID NO: 28 - DNA, 675 bp, Artificial Sequence
  codon-optimized endotope sequence
actagtgccaccatgatggaccaagctagatccgccttcagcaatctgttcggaggagagcccctctcctatacaagattctccctggccaggcaagtggacggcgacaactcccacgtcgagatgaaactcgccgtggatgaagaggagaacgccgacaataacaccaaggccaacgtgaccaagcctaagaggtgcagcggaagcatctgctacggcacaatcgccgtgatcgtcttcttcctgatcggattcatgatcggatacctgggctactgcaagggcgtggagcctaaaaccgagtgcgagagactcgctggaacagagtcccctgtcagggaggaacctggagaggatttccctgccccgcggtgcggatcccatctggtcgaagccctgtacctggtctgtggcgaggaaggattcttcaagagggctgtcaggcctctgtgggtgaggatggaaaagagatccctgaaaaaaggcgccgctgccctgggaattggcaccgactccgtcattctcatcgagggcagaggatccctcctgacctgtggcgacgtggaggaaaaccccggacccgaagctctgtacctggtgtgtggcgaaaggggctttttcctgtccgtctacctgaaaaccaatgtctttctgtttctgcccgggtaggtcgac

SEQ ID NO: 29 - protein, 218 aa, Artificial Sequence
  endotope sequence
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAPRCGSHLVEALYLVCGEEGFFKRAVRPLWVRMEKRSLKKGAAALGIGTDSVILIEGRGSLLTCGDVEENPGPEALYLVCGERGFFLSVYLKTNVFLFLPG

SEQ ID NO: 30 - DNA, 57 bp, Artificial Sequence
  Ins2B:9-23
tgtggttcccacctggtggaggctctctacctggtgtgtggggagcgtggcttcttc

SEQ ID NO: 31 - protein, 19 aa, Artificial Sequence
  Ins2B:9-23
CGSHLVEALYLVCGERGFF

SEQ ID NO: 32 - DNA, 57 bp, Artificial Sequence
  Ins2B:9-23 R22E
tgtggttcccacctggtggaggctctctacctggtgtgtggggaggagggcttcttc

SEQ ID NO: 33 - protein, 19 aa, Artificial Sequence
  Ins2B:9-23 R22E
CGSHLVEALYLVCGEEGFF

SEQ ID NO: 34 - DNA, 57 bp, Artificial Sequence
  Ins2B:9-23 E21G/R22E
tgtggttcccacctggtggaggctctctacctggtgtgtgggggagagggcttcttc

SEQ ID NO: 35 - protein, 19 aa, Artificial Sequence
  Ins2B:9-23 E21G/R22E
CGSHLVEALYLVCGGEGFF

SEQ ID NO: 36 - DNA, 54 bp, Artificial Sequence
  ChgA WE14
aagcgatggagcaggatggaccagctggccaaagagctgacagcagagaagcgg

SEQ ID NO: 37 - protein, 18 aa, Artificial Sequence
  ChgA WE14
KRWSRMDQLAKELTAEKR

SEQ ID NO: 38 - DNA, 42 bp, Artificial Sequence
  ChgA 1040-79
aagcgagcagttcgacctctatgggtacgtatggaaaagcgg

SEQ ID NO: 39 - protein, 14 aa, Artificial Sequence
  ChgA 1040-79
KRAVRPLWVRMEKR

SEQ ID NO: 40 - DNA, 57 bp, Artificial Sequence
  GAD65
tctctcaagaagggagctgcagccttagggattggaacagacagtgtgattctgatt

SEQ ID NO: 41 - protein, 19 aa, Artificial Sequence
  GAD65
SLKKGAAALGIGTDSVILI

SEQ ID NO: 42 - DNA, 72 bp, Artificial Sequence
  GAD65-524-543
agaatgagccgcctctcaaaggtggcgccagtgattaaagccagaatgatggagtatgggaccacaatggtc

SEQ ID NO: 43 - protein, 24 aa, Artificial Sequence
  GAD65-524-543
RMSRLSKVAPVIKARMMEYGTTMV

SEQ ID NO: 44 - DNA, 57 bp, Artificial Sequence
  P2A (from porcine teschovirus-1)
gctactaacttcagcctgctgaagcaggctggagacgtggaggagaaccctggacct

SEQ ID NO: 45 - protein, 19 aa, Artificial Sequence
  P2A (from porcine teschovirus-1)
ATNFSLLKQAGDVEENPGP

SEQ ID NO: 46 - DNA, 54 bp, Artificial Sequence
  T2A (from Thosea asigna virus)
gagggcagaggaagtctgctaacatgcggtgacgtcgaggagaatcctggacct

SEQ ID NO: 47 - protein, 18 aa, Artificial Sequence
  T2A (from Thosea asigna virus)
EGRGSLLTCGDVEENPGP

SEQ ID NO: 48 - DNA, 60 bp, Artificial Sequence
  E2A (from equine rhinitis A virus)
cagtgtactaattatgctctcttgaaattggctggagatgttgagagcaaccctggacct

SEQ ID NO: 49 - protein, 20 aa, Artificial Sequence
  E2A (from equine rhinitis A virus)
QCTNYALLKLAGDVESNPGP

SEQ ID NO: 50 - DNA, 66 bp, Artificial Sequence
  F2A (from Foot-and-mouth disease virus)
gtgaaacagactttgaattttgaccttctcaagttggcgggagacgtggagtccaaccctggacct

SEQ ID NO: 51 - protein, 22 aa, Artificial Sequence
  F2A (from Foot-and-mouth disease virus)
VKQTLNFDLLKLAGDVESNPGP

SEQ ID NO: 52 - DNA, 39 bp, Artificial Sequence
  Ins2B:15-23
gaggctctctacctggtgtgtggggagcgtggcttcttc

SEQ ID NO: 53 - protein, 13 aa, Artificial Sequence
  Ins2B:15-23
EALYLVCGERGFF

SEQ ID NO: 54 - DNA, 39 bp, Artificial Sequence
  IGRP-206-214
ttgagtgtgtacctgaagaccaacgtcttcctcttcctg

SEQ ID NO: 55 - protein, 13 aa, Artificial Sequence
  IGRP-206-214
LSVYLKTNVFLFL

SEQ ID NO: 56 - protein, 9 aa, Artificial Sequence
  GAD65-206-214
TYEIAPVFV

SEQ ID NO: 57 - protein, 9 aa, Artificial Sequence
  GAD65-546-554
SYQPLGDKV

SEQ ID NO: 58 - protein, 11 aa, Artificial Sequence
  hybrid insulin peptide
GDLQTLWSRMD

SEQ ID NO: 59 - protein, 11 aa, Artificial Sequence
  hybrid insulin peptide
LQTLALWSRMD

SEQ ID NO: 60 - protein, 11 aa, Artificial Sequence
  hybrid insulin peptide
LQTLALNAARD

SEQ ID NO: 61 - protein, 15 aa, Artificial Sequence
  hybrid insulin peptide
GQVELGGGSSPETLI

SEQ ID NO: 62 - protein, 15 aa, Artificial Sequence
  hybrid insulin peptide
GQVELGGGNAVEVLK